(19) United States
(12) Patent Application Publication
(10) Pub. No.: US 2022/0196635 A1
(43) Pub. Date: Jun. 23, 2022

(54) Fibrosis Assay

(71) Applicant: Georgia Tech Research Corporation, Atlanta, GA (US)

(72) Inventors: Stephen Robinson, Atlanta, GA (US); Jonathan Chang, Atlanta, GA (US); Shuichi Takayama, Atlanta, GA (US)

(21) Appl. No.: 17/514,264

(22) Filed: Oct. 29, 2021

Related U.S. Application Data
(63) Continuation of application No. 17/349,297, filed on Jun. 16, 2021, now abandoned.
(60) Provisional application No. 63/039,736, filed on Jun. 16, 2020.

(51) Int. Cl. G01N 33/50; G01N 1/30; C12N 5/077; C12M 3/06; C12M 1/40; C12M 1/00; C12N 9/74

(52) U.S. Cl. CPC G01N 33/5044; G01N 1/30; C12N 5/0656; C12M 23/16; C12M 21/18; C12N 2533/56; C12N 9/6429; C12Y 304/21005; G01N 2800/7052; G01N 2800/52; C12N 2533/54; C12M 23/22

(57) ABSTRACT

An exemplary embodiment of the present disclosure provides A method and system for forming a microscale cell-laden matrix using an aqueous two-phase system (“ATPS”) comprising a mixture of a first material and a second material having a phase boundary between the first and second materials. The method can comprise mixing an enzyme with the first material, mixing a protein with the second material, and mixing a suspension comprising cells with one of the first material or the second material, wherein the enzyme, protein, and suspension comprising cells generate the cell-laden matrix and wherein the first material comprises a first polymer comprising polyethylene glycol and the second material can be a second polymer selected from the group consisting of dextran, polyvinyl pyrrolidone, polyvinyl alcohol, or ficoll.

### CROSS-REFERENCE TO RELATED APPLICATIONS

This application is a continuation application of U.S. patent application Ser. No. 17/349,297, filed on 16 Jun. 2021, which claims the benefit of U.S. Provisional Application Ser. No. 63/039,736, filed on 16 Jun. 2020, which is incorporated herein by reference in its entirety as if fully set forth below.

### FEDERALLY SPONSORED RESEARCH STATEMENT

This invention was made with government support under grant/award number R21AG061687 awarded by the National Institutes of Health, grant/award number R01HL136141, awarded by the National Institutes of Health, and N66001-13-C-2027, awarded by the Department of the Navy. The government has certain rights in the invention.

### FIELD OF THE DISCLOSURE

The various embodiments of the present disclosure relate generally to methods and systems for forming assays of microscale cell-laden matrices, and more particularly to fibrin assays formed using an aqueous two-phase system.

### BACKGROUND

Studying the process of remodeling events such as tissue degradation or formation at the cellular level can provide valuable information to the overall process of wound healing. Even more so, the differences between normal and pathogenic wound healing of various tissues has broad applications in studying many different cell types and diseases. In particular, following tissue damage, fibrin will form a temporary scaffold at the injury that enables fibroblasts to migrate to the site for matrix remodeling. The fibrotic remodeling events that occur after can result in a variety of complications, for example, excessive collagen accumulation promoting fibrotic scarring.

Current approaches available to evaluate such fibrotic remodeling events require large volumes that hinder high-throughput adaption or fail to consider key contributing factors such as specific environmental factors, epigenetics, or senescence. An approach that can control the crosslinking process within fibrotic systems by separating the enzyme, thrombin, and the protein, fibrinogen, in separate and distinct phases can form microscale matrices for high-throughput screening for fibrosis and other dysregulated wound healing diseases.

### BRIEF SUMMARY

The present disclosure relates to methods for forming microscale cell-laden matrices and systems for making an assay having microscale cell-laden matrices. An exemplary embodiment of the present disclosure provides a method for forming a microscale cell-laden matrix using an aqueous two-phase system (“ATPS”) comprising a mixture of a first material and a second material having a phase boundary between the first and second materials. The method can comprise mixing an enzyme with the first material, mixing a protein with the second material, and mixing a suspension comprising cells with the first material. The enzyme, protein, and suspension can comprise cells to generate the cell-laden matrix. The first material can be a first polymer selected from the group consisting of polyethylene glycol, polyvinyl pyrrolidone, polyvinyl alcohol, and ficoll. The second material can be a second polymer comprising dextran, polyvinyl pyrrolidone, polyvinyl alcohol, and ficoll. The second material can be selected to be different from the first material.

In any of the embodiments disclosed herein, at least one of the enzyme or the cells in the first material can be configured to diffuse into the second material.

In any of the embodiments disclosed herein, the mixture can comprise up to about 300 microliters (μL) of volume.

In any of the embodiments disclosed herein, the enzyme can comprise a plasma enzyme from the group consisting of prothrombin, thrombin, amylase, pepsin, lipoprotein lipase, and pseudo-choline esterase.

In any of the embodiments disclosed herein, the protein can comprise a plasma protein from the group consisting of fibrinogen, fibronectin, collagen, albumin, globulin, and plasminogen activator inhibitor type 1.

In any of the embodiments disclosed herein, the suspension comprising cells can comprise fibroblasts, fibrocytes, osteoblasts, myofibroblasts, epithelial cells, endothelial cells, immune cells, mesenchymal cells, cancer cells, and stem cells.

In any of the embodiments disclosed herein, the method can further comprise mixing the mixture with a third material comprising one or more additives.

In any of the embodiments disclosed herein, the method can further comprise imaging the cell-laden matrix and the one or more additives.

In any of the embodiments disclosed herein, the one or more additives can comprise transforming growth factor beta 1 (TGF-β1).

In any of the embodiments disclosed herein, the method can further comprise adding, to the cell-laden matrix, a digestive agent.

In any of the embodiments disclosed herein, the method can further comprise imaging the cell-laden matrix and the digestive agent.

In any of the embodiments disclosed herein, the method can further comprise detecting one or more remodeling events of the cell-laden matrix selected from the group consisting of matrix degradation, matrix growth, matrix proliferation, matrix cell invasion, matrix cell contraction, matrix cell type, and matrix cell density.

An exemplary embodiment of the present disclosure provides a cell-laden matrix assay system comprising a solid support comprising at least one defined area and an aqueous two-phase system mixture for forming a cell-laden matrix. The mixture can comprise a first material having an enzyme and one or more cells, a second material having a protein, and a phase boundary between the first and second materials. The first material can be a first polymer selected from the group consisting of polyethylene glycol, polyvinyl pyrrolidone, polyvinyl alcohol, and ficoll. The second material can be a second polymer comprising dextran, polyvinyl pyrrolidone, polyvinyl alcohol, and ficoll. The second material can be selected to be different from the first material.

In any of the embodiments disclosed herein, the at least one defined area can comprise up to about 300 microliters (μL) of volume.

In any of the embodiments disclosed herein, forming the cell-laden matrix can comprise the enzyme, the protein, and at least one cell.

In any of the embodiments disclosed herein, said solid support can be selected from the group consisting of a plate, a multiwell plate, a microfluidic device, and a slide.

In any of the embodiments disclosed herein, the system can further comprise a third material comprising one or more additives.

In any of the embodiments disclosed herein, the system can further comprise a digestive agent.

In any of the embodiments disclosed herein, the system can further comprise a detection system selected from the group consisting of label-free image processing, colorimetric, fluorescent, fluorescence polarization or lifetime readings, refractive index change, and electrochemical detection systems.

In any of the embodiments disclosed herein, the enzyme can comprise a plasma enzyme from the group consisting of prothrombin, thrombin, amylase, pepsin, lipoprotein lipase, and pseudo-choline esterase. The protein can comprise a plasma protein from the group consisting of fibrinogen, fibronectin, collagen, albumin, globulin, and plasminogen activator inhibitor type 1. The one or more cells can comprise a fibroblast, a fibrocyte, an osteoblast, a myofibroblast, an epithelial cell, a mesenchymal cell, a cancer cell, and a stem cell.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A through 1D provide aqueous two-phase system (ATPS) fibrin printing and cell-mediated degradation, in accordance with an exemplary embodiment of the present invention.

FIGS. 2A through 2E provide high-throughput quantification of fibrin degradation, in accordance with an exemplary embodiment of the present invention.

FIGS. 3A through 3F provides assay volume consistency, in accordance with an exemplary embodiment of the present invention.

FIGS. 4A through 4F provide cell density and TGF-β1 effects, in accordance with an exemplary embodiment of the present invention.

FIGS. 5A through 5H provide cell donor and drug stimulation, in accordance with an exemplary embodiment of the present invention.

FIGS. 6A through 6D provide ATPS fibrin printing and cell-mediated remodeling, in accordance with an exemplary embodiment of the present invention.

FIGS. 7A through 7H provides Matrix remodeling in vitro, in accordance with an exemplary embodiment of the present invention.

FIGS. 8A through 8C provide TGF-β1, fetal bovine serum concentration, and seeding density effects, in accordance with an exemplary embodiment of the present invention.

FIGS. 9A through 9K show consistency in response between cell lines, in accordance with an exemplary embodiment of the present invention.

FIGS. 10A through 10D provide response to therapeutic stimuli, in accordance with an exemplary embodiment of the present invention.

FIGS. 11A through 11F provide high-throughput quantification of fibrin remodeling, in accordance with an exemplary embodiment of the present invention.

FIGS. 12A through 12C show TGF-β1, serum concentration, and seeding density effects, in accordance with an exemplary embodiment of the present invention.

FIGS. 13A through 13C show response to therapeutic stimuli, in accordance with an exemplary embodiment of the present invention.

FIG. 14 provides ECM remodeling, in accordance with an exemplary embodiment of the present invention.

FIG. 15 provides a plot comparing fibrin degradation between different age animal subjects, in accordance with an exemplary embodiment of the present invention.

FIG. 16 provides an exemplary method for forming a microscale cell-laden matrix, in accordance with an exemplary embodiment of the present invention.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

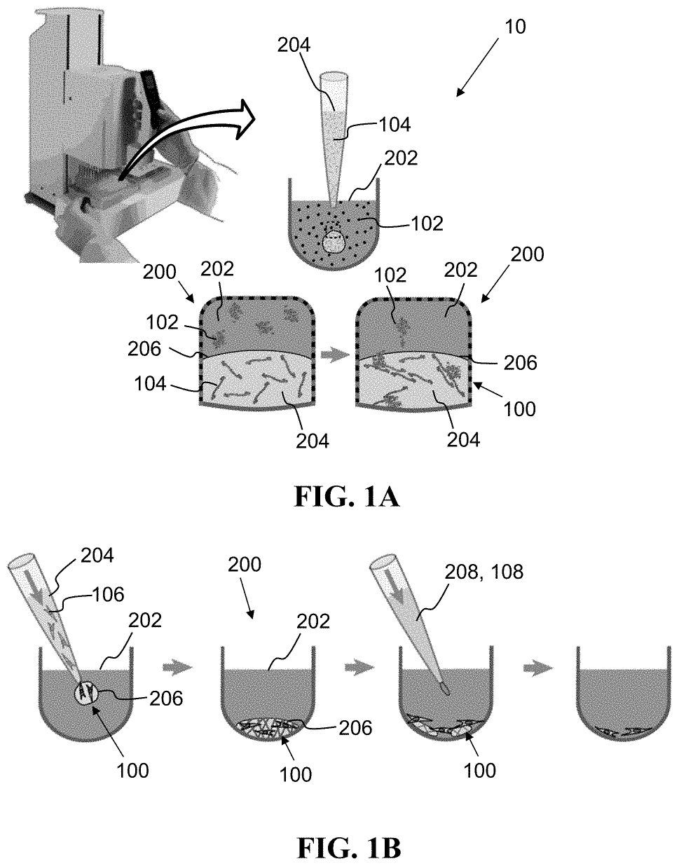

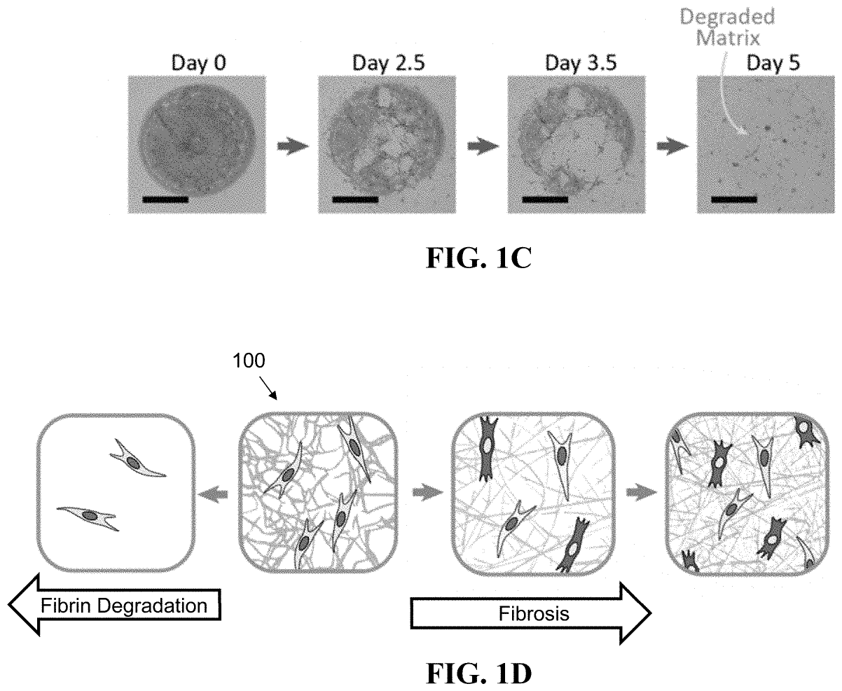

As shown in FIG. 1A, an exemplary embodiment of the present invention provides a method 10 for forming a cell-laden matrix 100 where an aqueous two-phase system (“ATPS”) 200 is used to control mixing of an enzyme 102 in a first material 202 with a protein 104 in a second material 204. Between the first a second materials 202, 204, a phase boundary 206 can form when the two materials are immiscible. In general, FIG. 1A illustrates the enzymatic control enabled by ATPS printing of fibrin scaffolds, whereby thrombin from the PEG phase diffuses into the DEX phase and crosslinks the fibrinogen into fibrin during the incubation period. FIG. 1B provides a schematic of ATPS generation of microscale fibrin droplets and subsequent fibrinolysis. FIG. 1C provides characteristic brightfield microscope images (taken at 4× magnification) showing the assay progression when stimulated with 0.5 ng/mL of TGF-β1, showing an opaque fibrin matrix and progressive degradation with scale bars of 1 mm. FIG. 1D provides a schematic of two example remodeling events including cell-mediated fibrinolysis (left) or concurrent fibrinolysis and collagen deposition. In some examples, collagen deposition benefits from serum and higher cell density.

As used herein, the first material 202 and the second material 204, which constitute the aqueous two-phase system (“ATPS”) 200, can include any suitable polymer/polymer ATPS system. Polymer/polymer ATPS systems can include two nonionic polymers, such as, polyethylene glycol (PEG) mixed with any one of polypropylene glycol (PPG), polyvinyl pyrrolidone (PVP), poly(vinyl methyl ether) (PVME), poly(vinyl methyl ethyl ether) (PVMEE) polyether sulfones (PES), polyvinyl alcohol (PVA), polypropylene glycol dimethyl ether (PPGDME), UCON, Ficoll, Dextran, Pullulan, Maltodextrin, and hydroxypropyl starch. As an alternative, a two nonionic polymer ATPS system may also include dextran mixed with any one of PVP, PVA, PPG, UCON, Ficoll, hydroxypropyl starch, and Natrosol. In some embodiments, a two nonionic polymer ATPS system may include PPG mixed with any one of polyethylene glycol methyl ether (PEGME), polyethylene glycol dimethyl ether (PEGDME), PVA, and Ficoll. In some embodiments, the polymer/polymer ATPS systems can include one nonionic polymer and one ionic polymer such as PEG/dextran sulphate, PEG/polyacrylic acid (PAA), PEG/polyacrylamide (PAM), PEG/carboxymethyl dextran, PVP/PAM, and PVA/acrylic polymers. In any of the embodiments herein, the polymer/polymer ATPS system can include two ionic polymers such as dextran sulphate/polystyrene sulfonate (PSS) or dextran sulphate/diethylaminoethanol (DEAE)-dextran. In addition, suitable ATPS systems may include but are not particularly limited to, water/ethylene oxide propylene oxide (EOPO), PEG/high-concentration salt, PEG/levan, PEG/ammonium sulfate, PEG/sodium sulfate, PEG/magnesium sulfate, PEG/potassium phosphate, and PEG/sodium carbonate.

The two materials used to form the aqueous two-phase system 200 are preferably polyethylene glycol/dextran. In some embodiments, the enzyme 102 can be mixed in the polyethylene glycol phase and the protein 104 may be characterized by being concentrated in the dextran phase, and the proteins 104 concentrated in the dextran phase may be isolated using a pipette, or other suitable methods. As an alternative, the enzyme 102 can be concentrated in the dextran phase while the protein 104 can be mixed in the polyethylene glycol phase. In certain embodiments, additives can also be included in either the polyethylene glycol phase or the dextran phase.

As used herein, the enzyme 102 can include any substance composed wholly or largely of protein or polypeptides that catalyzes or promotes, more or less specifically, one or more chemical or biochemical reactions. In certain embodiments, the ATPS 200 can include one or more enzymes from blood plasma or other bodily fluids. Suitable plasma enzymes can include, but are not limited to, prothrombin, thrombin, amylase, pepsin, lipoprotein lipase, and pseudo-choline esterase. In general, thrombin is an activated enzyme, also known as α-thrombin, which results from the proteolytic cleavage of prothrombin (factor II). As an alternative, or in addition thereto, the enzyme may include cell types that produce endogenous prothrombin.

In some embodiments, the ATPS 200 can include one or more proteins 104, including soluble proteins found in the plasma of normal humans or animals. These include but are not limited to coagulation proteins, albumin, lipoproteins and complement proteins. In particular, plasma proteins can include fibrinogen, albumin, globulin, and plasminogen activator inhibitor type 1.

Referring back to FIG. 1B, the method 10 can further include mixing a suspension of cells 106 with the first material 202 by including it in the second material 204 with the protein 104. Alternatively, the suspension of cells 106 can be mixed with the first material 202 in a separate addition step from mixing the protein 104 with the first material 202. In any of the embodiments disclosed herein, mixing the suspension of cells 106 can be done in multiple steps, for instance, the suspension of cells 106 can be mixed after the first material 202 and the second material 204 have been mixed. Alternatively, or in addition thereto, the suspension of cells 106 can be added to the mixture after the first and second materials 202, 204 have mixed and the enzyme 102 and the protein 104 have been mixed. In certain embodiments, after the first and second materials 202, 204 have been mixed, the mixture can be washed in one or more washing cycles prior to the addition of the suspension of cells 106.

In some embodiments, the cell suspension 106 can include cells that can form matrixes when mixed with the enzyme 102 and the protein 104. Cell suspension 106 can be made up of a variety of cells found in normal humans or animals. Suitable cells for forming matrixes can include, but are not limited to fibroblasts, fibrocytes, osteoblasts, myofibroblasts, epithelial cells, endothelial cells, immune cells, mesenchymal cells, cancer cells, and stem cells. In certain embodiments, cell suspension 106 can include cells from a particular subject. For instance, studying fibrosis on a subject that has been exposed to smoking can include mixing human lung fibroblasts from a subject with a history of smoking. Such ATPS assay can provide insight into idiopathic pulmonary fibrosis.

Mixing the enzyme 102, the protein 104, and cell suspension 106 can generate the cell-laden matrix 100. In certain embodiments, mixing thrombin with fibrinogen and fibroblasts can result in a fibrin cell-laden matrix. As would be appreciated, changing the enzyme 102, protein 104, or cell suspension 106 can generate a variety of cell-laden matrices using an ATPS mixture.

In some embodiments, using method 10 to create an ATPS assay of a subject may include collecting body fluid from a subject to study a disease. Here, the body fluid may include, but is not particularly limited to, at least one selected from the group consisting of whole blood, serum, peritoneal fluid, breast milk, and urine. The disease may include, but is not particularly limited to, at least one selected from the group consisting of fibrosis, pulmonary fibrosis, cancer, sepsis, arteriosclerosis, rheumatoid arthritis, dermatomyositis, polymyositis, mixed connective tissue disease, systemic lupus erythematosus, sarcoidosis, scleroderma, and pneumonia.

Referring back to FIG. 1B, method 10 can further include adding to the ATPS mixture a third material 208 having one or more additives 108. In some embodiments, the third material 208 can include, without limitation, media, media free of polyethylene glycol, or protein degradation material (e.g., plasminogen-degradation material). The third material 208 can be concentrated with one or more additives 108 such as, for example, extracellular matrix (ECM), collagen, plasminogen, TGF-β1, drugs, serums (e.g., fetal bovine serum, newborn calf serum, bovine calf serum, iron supplemented calf serum, fetalgo, cosmic calf serum, and fetalclone III serum), cytokines, and hormones. In some embodiments, additives can be added to the cell-laden matrix 100 in order to degrade the matrix, as shown via brightfield microscope images in FIG. 1C. Additives for degrading the cell-laden matrix 100 can include digestive agents such as, for example, plasminogen, plasmin, serine proteases, or other suitable digestive enzymes.

In some embodiments, a mixture of the first and second materials 202, 204 comprising the enzyme 102, the protein 104, and cell suspension 106 can comprise up to about 300 microliters of volume. Preferably, the mixture can range from about 0.5 μL to about 300 μL, and more preferably from 100 to 200 μL. In any of the embodiments herein, a volume of the first material comprising the enzyme 102 and optionally comprising the one or more additives 108 can be between about 50 and 200 μL mixed with a volume of the second material 204 comprising the protein 104 and optionally comprising the cell suspension 106 can be between about 0.5 μL and 50 μL, and preferably from 10 μL to about 50 μL.

FIG. 1D provides a schematic of potential remodeling events that can occur after the cell-laden matrix 100 is formed. Certain remodeling events can include without limitation matrix degradation, matrix grown, matrix proliferation, matrix cell invasion, matrix cell contraction, matrix cell type, and matrix cell density. In the example of a fibrin matrix, method 10 can be used to study fibrinolysis as well as collagen deposition, fibrotic pathogenesis, and the like.

In some embodiments, following formation of the cell-laden matrix 100, method 10 can further comprise imaging the cell-laden matrix 100 and the progression of cellular remodeling. Imaging techniques can include phase-contrast microscopy, fluorescent imaging, brightfield microscopy, confocal microscopy, 4D live-cell imaging (e.g., confocal with time-lapse microscopy), and other suitable cellular imaging techniques. In some embodiments, the imaging of matrix degradation can be achieved with label-free methods such as with absorbance or brightfield microscopy. As would be appreciated, such imaging methods can be automated by implementing a cellular imaging library in combination with artificial intelligence or machine learning methods.

In some embodiments, cellular remodeling events can also be detected by a detection system selected from the group consisting of label-free image processing, colorimetric, fluorescent, fluorescence polarization or lifetime readings, refractive index change, and electrochemical detection systems.

As would be appreciated, forming the cell-laden matrix can be done using a range of volumes while keeping ratios of the enzyme 102, protein 104, and cell suspension 106 the same or similar. In some embodiments, the cell-laden matrix can be formed from at least one cell within the cell suspension 106 such that the cell-laden matrix can be analyzed on a single-cellular level.

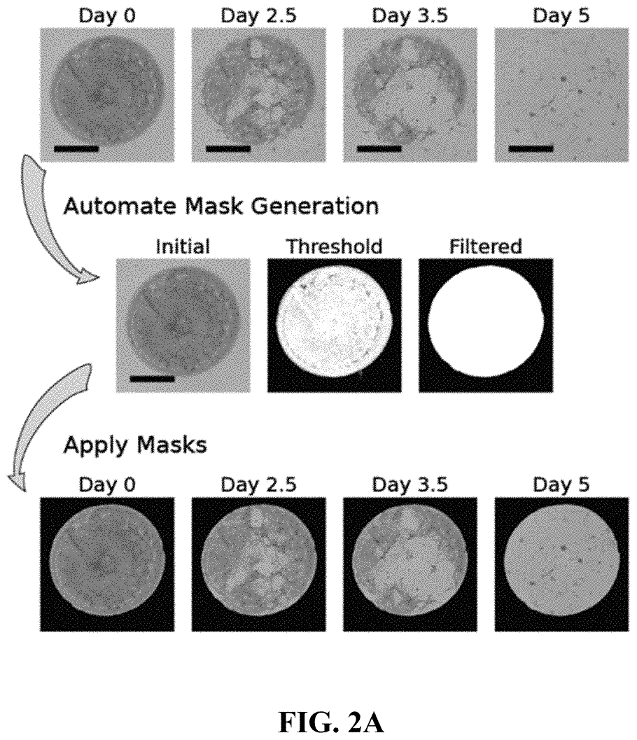

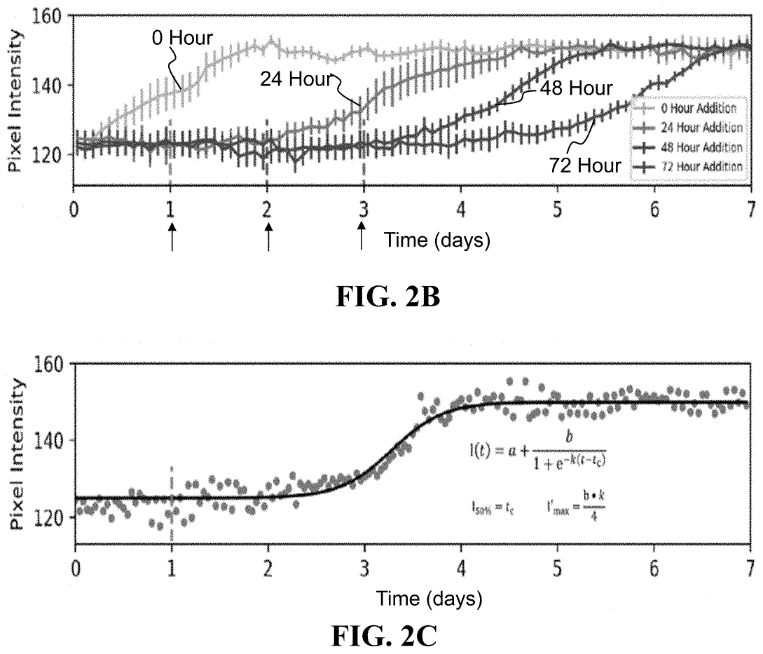

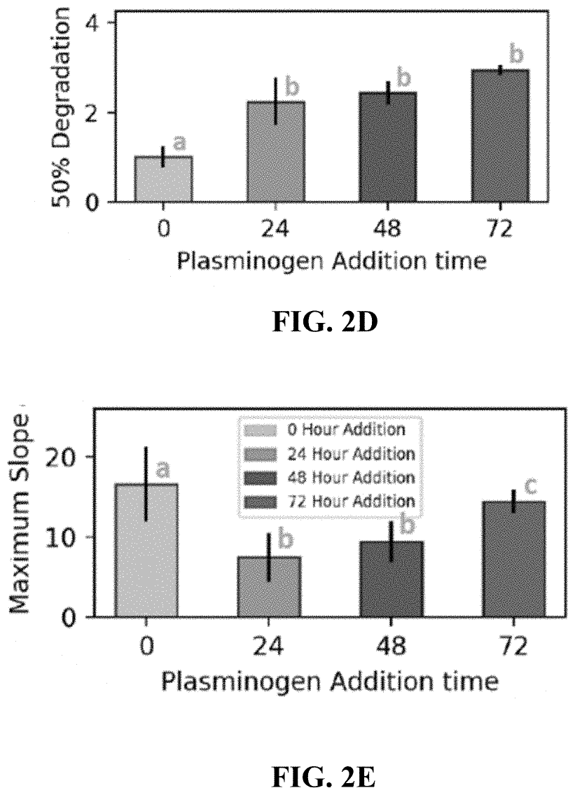

FIG. 2A shows automated image processing and analysis utilized a library for computer vision, machine learning, and image processing for thresholding and morphological filtering in order to establish an initial mask for each individual assay that was applied to all assay images for that well with scale bars of 1 mm. FIG. 2B provides a plot of time (days) versus pixel intensity, where the average pixel intensity within masked regions was plotted for time course evaluation with different plasminogen addition times indicated by arrows. FIG. 2C provides an example measurement demonstrating image metric extraction by fitting a logistic function to time course pixel intensity data with least squares regression. FIG. 2D shows the time point for 50% degradation. FIG. 2E shows the maximum slope from the sigmoid centroid, determined using logistic functions fit for each experimental replicate. Note that the 50% degradation time (vertical axis) is indicated as days after plasminogen addition, while the plasminogen addition time (horizontal axis) is in hours. (Statistical significance for (d, e) P<0.01 by ANOVA. ab=P<0.01; bc=P<0.05; ac=P<0.1 by post-hoc Tukey test. N=5 for all conditions.

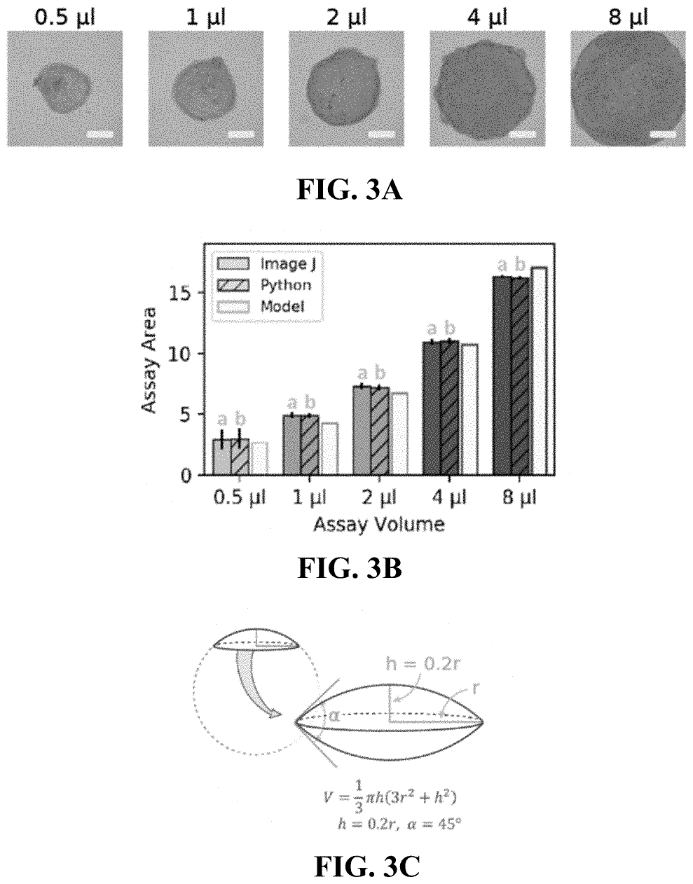

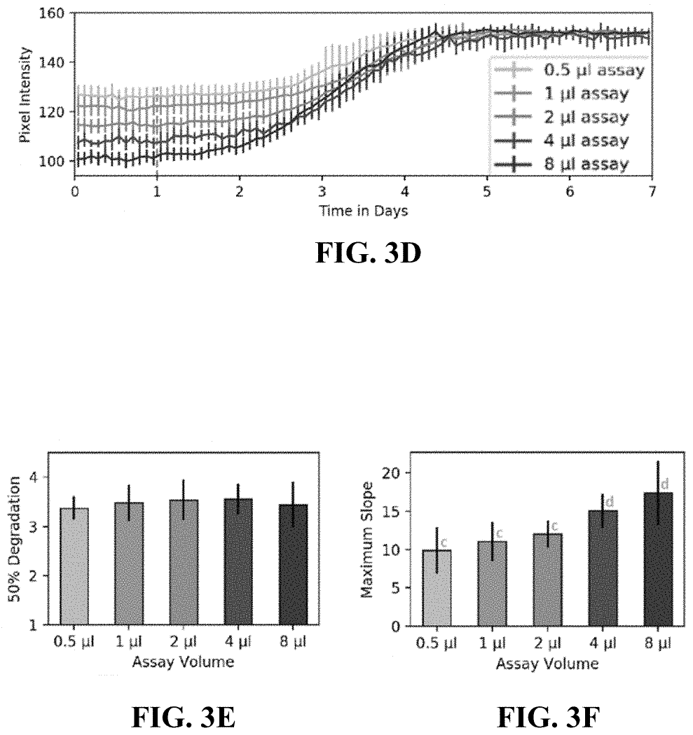

FIG. 3A shows ATPS printing of fibrin scaffolds demonstrated consistency in assay shape and texture between volumes with scale bars of 1 mm. FIG. 3B provides a plot of assay volume versus assay area demonstrating compared cross sectional area of assays between image J, Python generated masks, and a geometric model of assay volume. FIG. 3C is a schematic of a doubled spherical cap showing the best fit of the geometric volume models evaluated (including sphere, hemisphere, and single spherical cap). FIG. 3D provides a plot of time (days) versus pixel intensity showing changes in average pixel intensity for different assay volumes to demonstrate consistency in fibrin degradation time between volume conditions (different initial pixel intensity values between conditions indicate varied transmission of light through different volume constructs). FIG. 3E shows the 50% degradation time. FIG. 3F shows maximum slope. (Statistical significance for (b, f) P<0.01 by ANOVA. ab=P>0.2. cd=P<0.05 by post-hoc Tukey test. N=4 for all conditions).

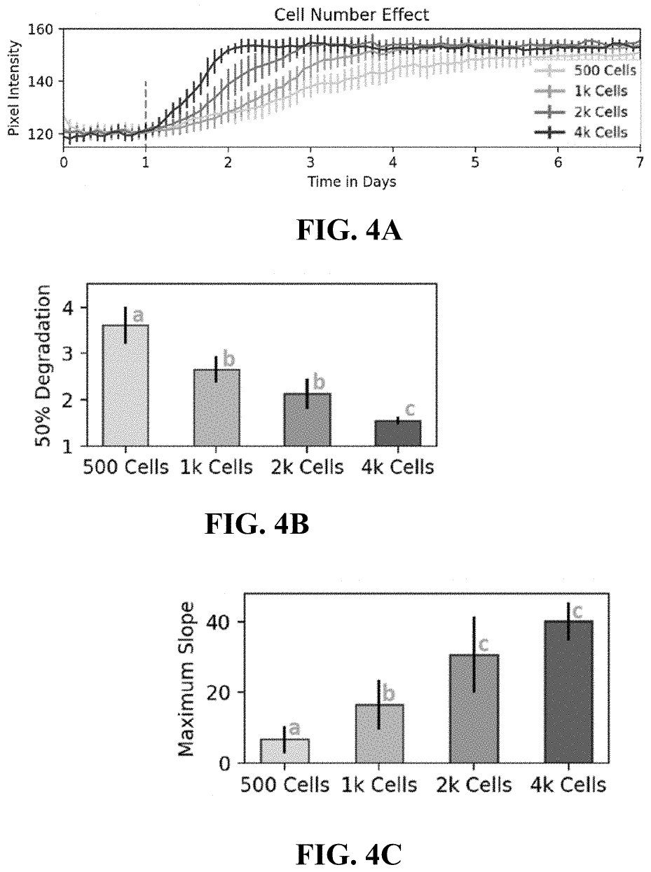

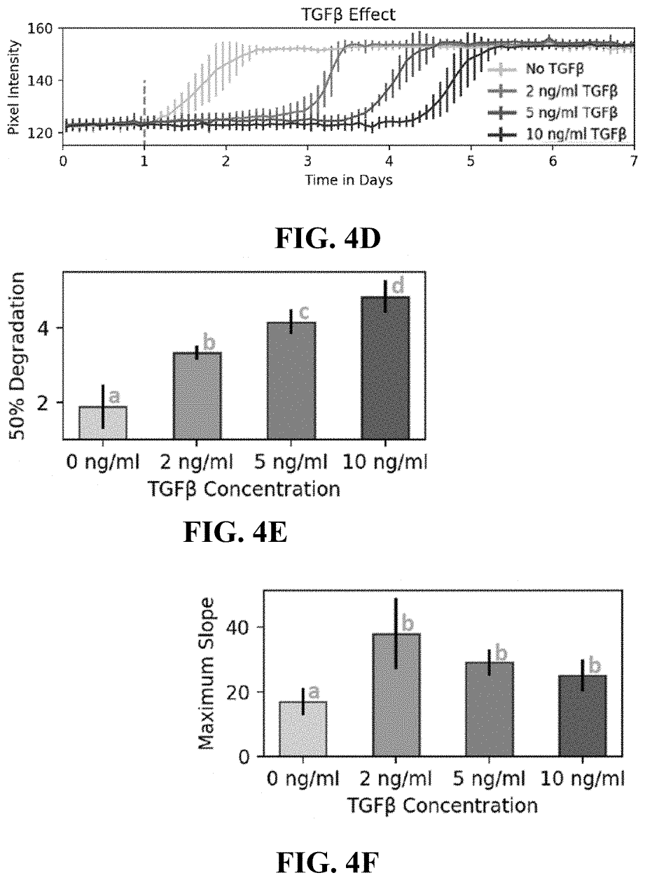

FIG. 4A provides a plot of time (days) versus pixel intensity showing changes in fibrin degradation between different densities of cells within a 1 μl assay. FIG. 4B shows the 50% degradation time demonstrating decreased fibrinolysis time and increased slope with higher cell counts. FIG. 4C shows the maximum slope demonstrating decreased fibrinolysis time and increased slope with higher cell counts. FIG. 4D provides a plot of time (days) versus pixel intensity for various concentrations of TGF-β1 indicating delays in fibrin degradation in response to the stimulus. FIG. 4E shows the 50% degradation time demonstrating increases in fibrinolysis time but no significant changes in slope with higher concentrations of TGF-β1. FIG. 4F shows the maximum slope demonstrating increases in fibrinolysis time but no significant changes in slope with higher concentrations of TGF-β1. (Statistical significance P<0.01 by ANOVA. In (b, c, e, f) P<0.05 by post-hoc Tukey test between all bars with different lettered labels. N=4 for all conditions).

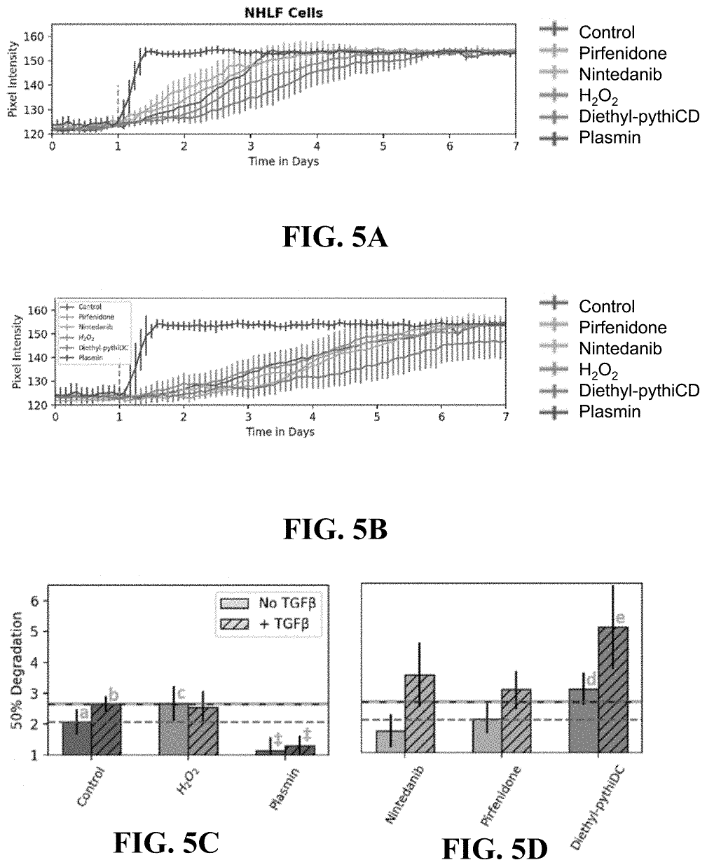

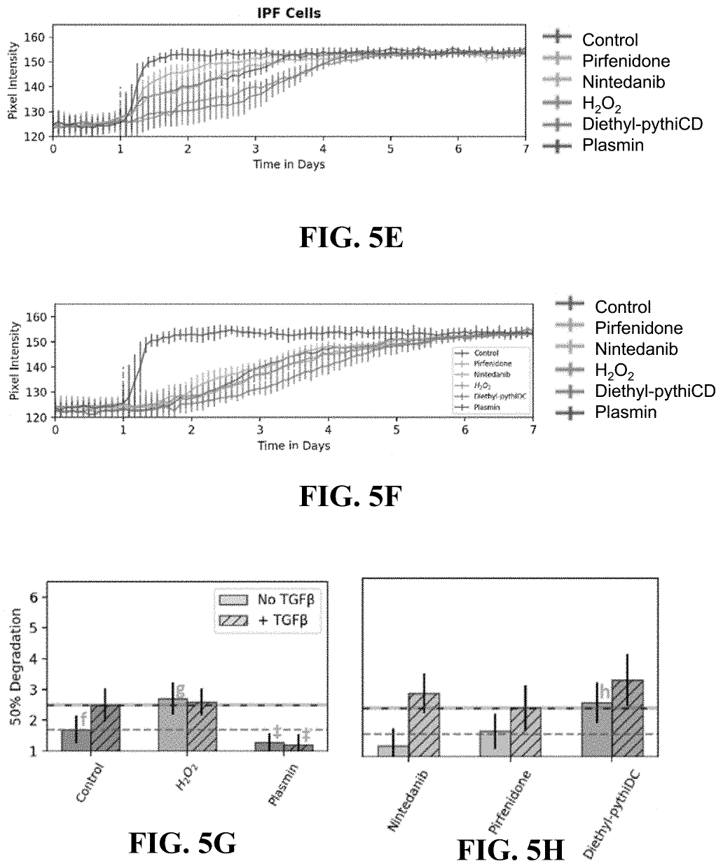

FIG. 5A provides a plot of time (days) versus pixel intensity showing the effects on fibrin degradation of several different stimulants with NHLF cells, but with no TGF-β1. FIG. 5B similarly shows the effects on fibrin degradation of several different stimulants with NHLF cells, but with 2 ng/ml TGF-β1. FIGS. 5C and 5D show the sigmoid fits used to determine 50% degradation time from FIGS. 5A and 5B. FIG. 5E provides a plot of time (days) versus pixel intensity showing the effects on fibrin degradation of several different stimulants with diseased IPF cells but with no TGF-β1. FIG. 5F similarly shows the effects on fibrin degradation of several different stimulants with diseased IPF cells, but with 2 ng/ml TGF-β1. FIGS. 5G and 5H show the sigmoid fits used to determine 50% degradation time from FIGS. 5A and 5B. Dotted lines show mean value from control conditions for comparison. (Statistical significance P<0.01 by two-way ANOVA: As the positive control, plasmin was excluded from ANOVA. ‡=P<0.01. ad, be=P<0.05. ac, fg, fh=P<0.1 by post-hoc Tukey test. N=4 for all conditions).

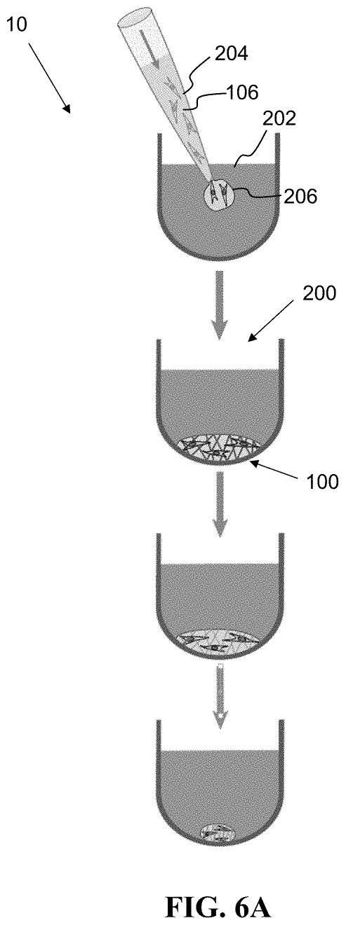

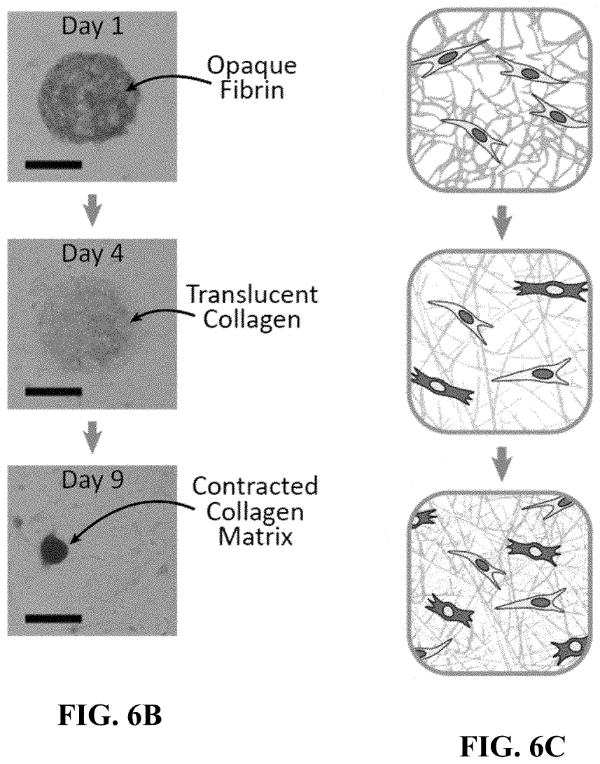

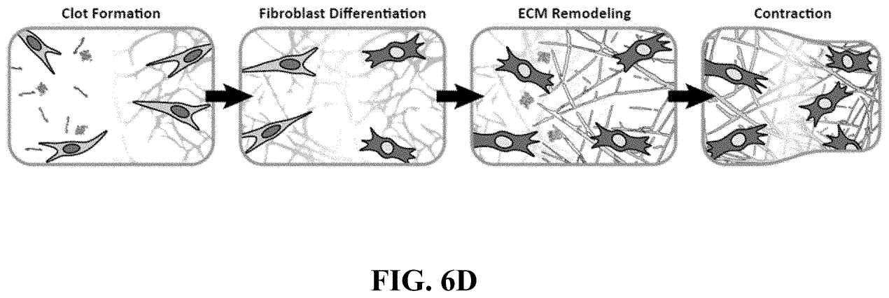

FIG. 6A illustrates a schematic of an example ATPS generation of microscale fibrin droplets and subsequent remodeling, showing thrombin from the PEG phase diffusing into the dextran phase for controlled conversion of fibrinogen into fibrin over the incubation period and subsequent remodeling including concurrent fibrinolysis, collagen deposition, and contraction. FIG. 6B shows characteristic brightfield microscope images (taken at 4× magnification) illustrate the assay progression when stimulated with 2 ng/mL of TGF-β1 with scale bars of 1 mm. FIG. 6C illustrates microscale schematics showing changes in ECM organization at stages of remodeling. Fibrosis denotes deposition and accumulation of fibrous extracellular protein. FIG. 6D provides a schematic of example fibroplasia assays and the stages of wound healing: Following tissue injury, the process of wound healing can be broken down into clot formation, fibroblast differentiation, ECM remodeling, and contraction. Aberrant progression of these steps can result in tissue fibrosis.

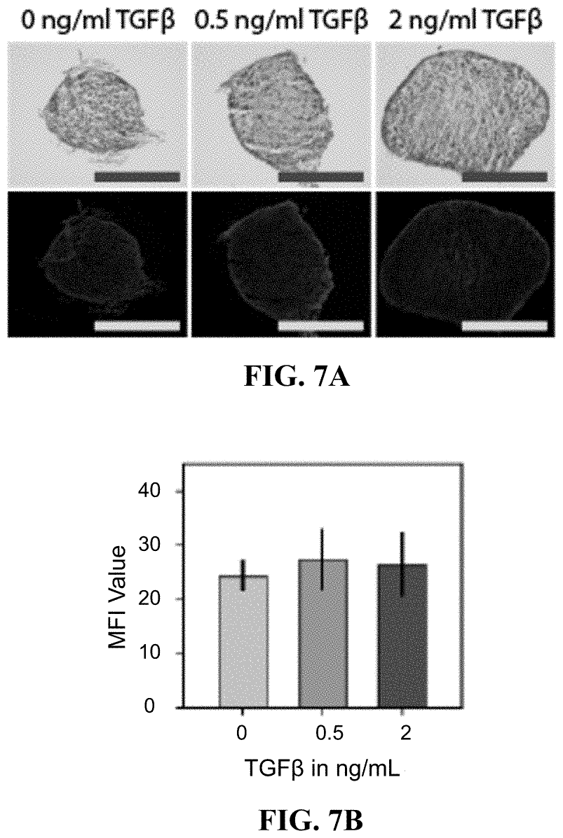

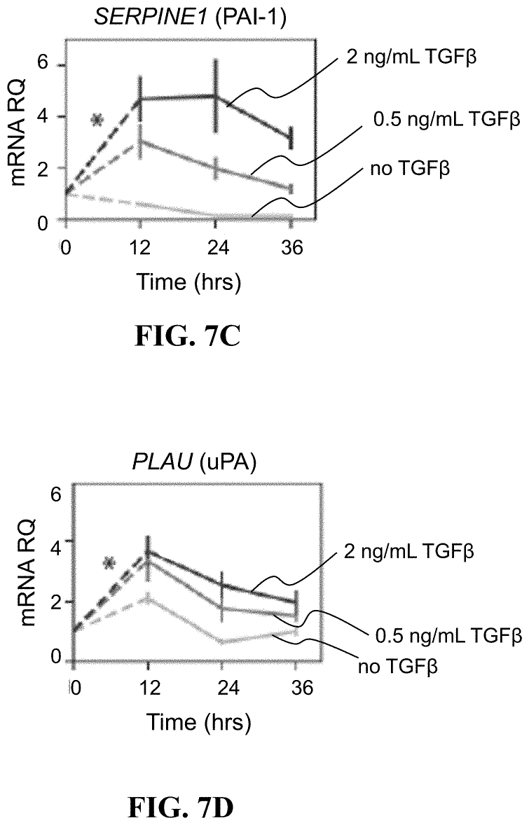

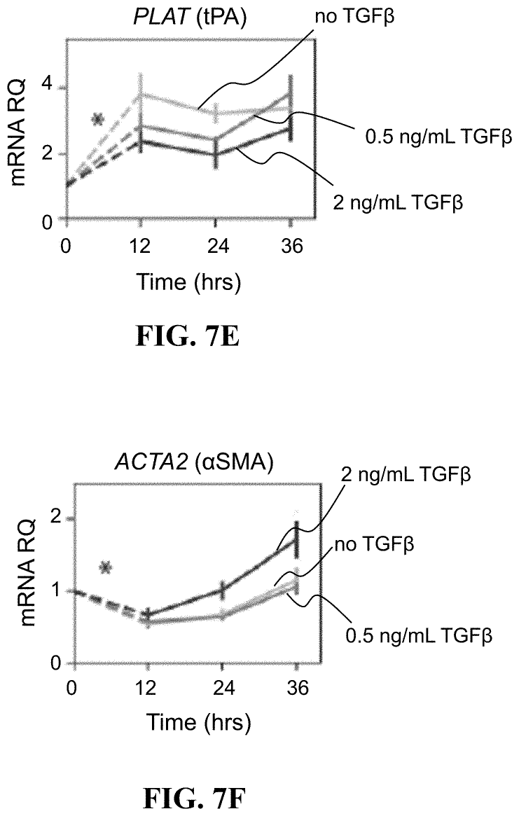

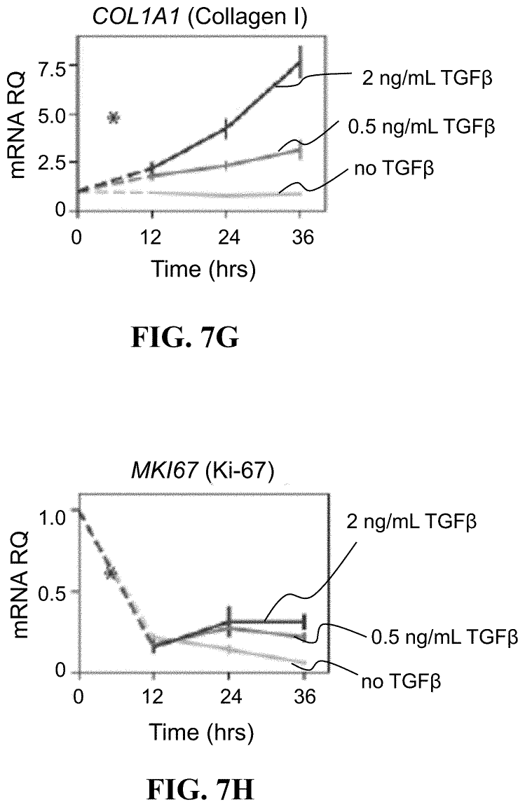

FIG. 7A shows brightfield images of histologic sections illustrating the difference in final size between assays treated with varied concentrations of TGF-β1. The contracted assays were harvested after 12 days, and sections were stained with picrosirius red. Scale bars are 250 μm. FIG. 7B provides evaluation of mean fluorescence intensity demonstrates consistency in collagen organization across conditions. FIG. 7C shows quantification of mRNA expression via qPCR evaluated dose-dependent time-course changes in SERPENE1 in response to concentrations of TGF-β1. FIG. 7D shows quantification of mRNA expression via qPCR evaluated dose-dependent time-course changes in COL1A1 in response to concentrations of TGF-β1. FIG. 7E shows quantification of mRNA expression via qPCR evaluated dose-dependent time-course changes in ACTA2 in response to concentrations of TGF-β1. FIG. 7F shows quantification of mRNA expression via qPCR evaluated dose-dependent time-course changes in PLAU in response to concentrations of TGF-β1. FIG. 7G shows quantification of mRNA expression via qPCR evaluated dose-dependent time-course changes in PLAT in response to concentrations of TGF-β1. FIG. 7H shows quantification of mRNA expression via qPCR evaluated dose-dependent time-course changes in MKI67 in response to concentrations of TGF-β1. The dotted lines indicate the zero-time point used as reference for relative expression. Two-way ANOVA indicated significant effects of time-point and TGF-β1 stimulation for SERPENE1, PLAU, PLAT, ACTA2, COL1A1, and MKI67 with P<0.05.

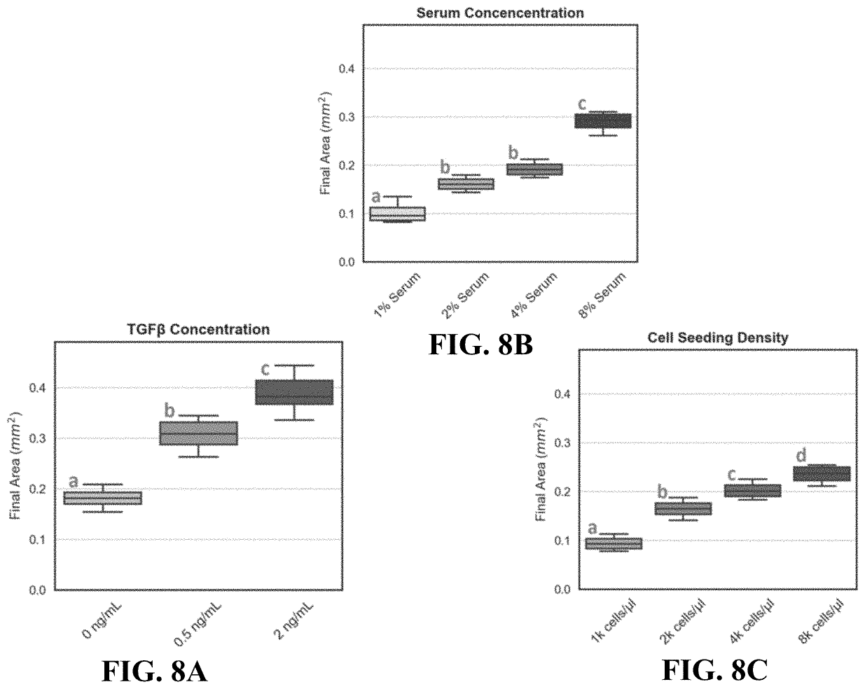

FIGS. 8A through 8C provide TGF-β1, fetal bovine serum concentration, and seeding density effects. In FIG. 8A, fibrin assays were evaluated with different TGF-β1 concentration. In FIG. 8B, fibrin assays were evaluated with different serum concentrations. In FIG. 8C, fibrin assays were evaluated with different cell seeding density to evaluate changes in final size. (Statistical significance by ANOVA: P<0.05; Post-hoc Tukey test: ab, bc, cd=P<0.05; N=6 for TGF-β1 conditions and N=4 for serum and cell seeding).

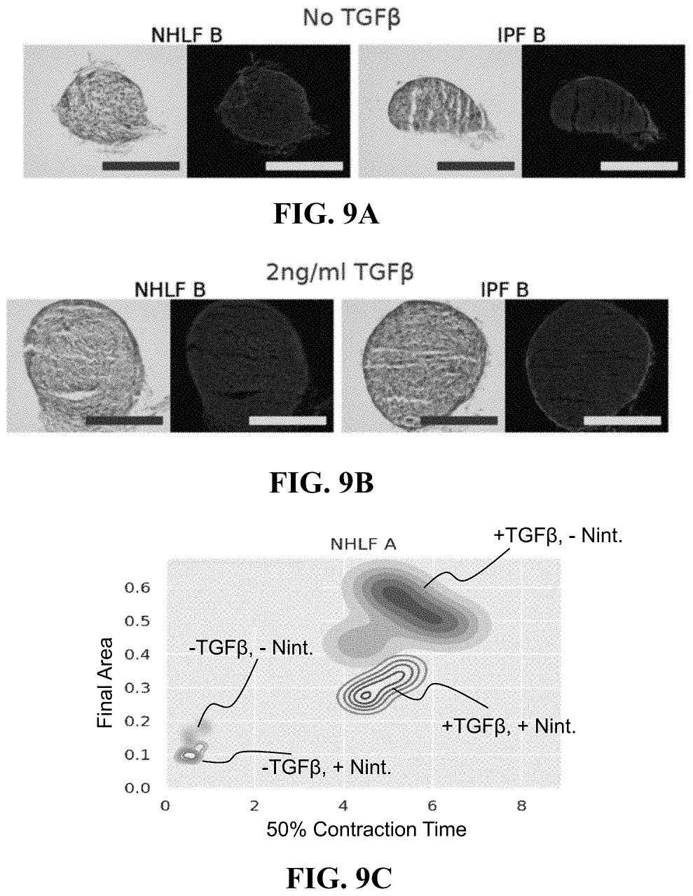

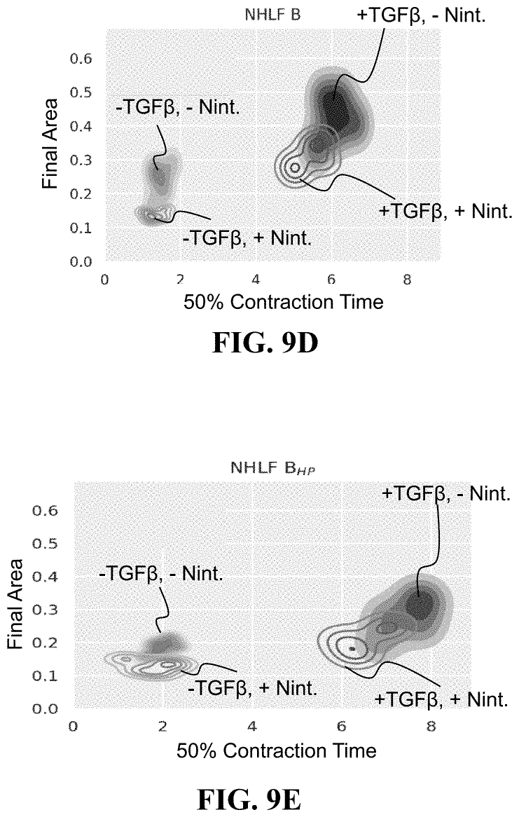

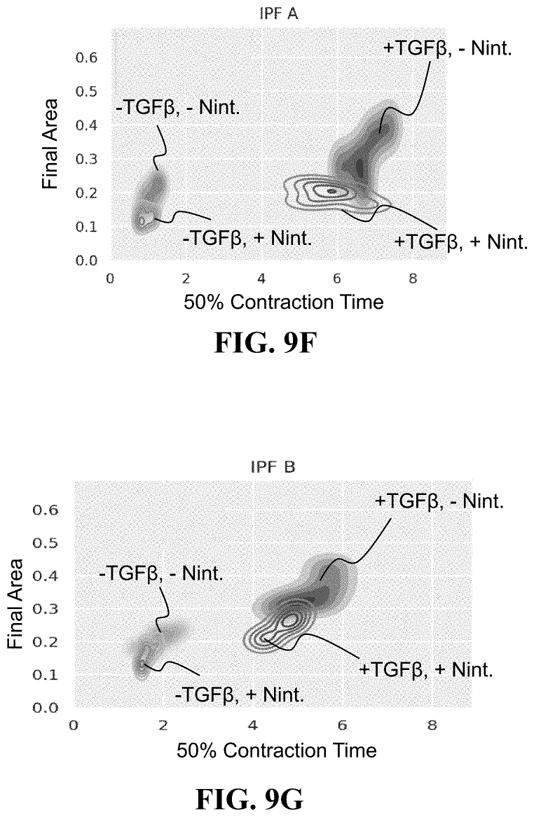

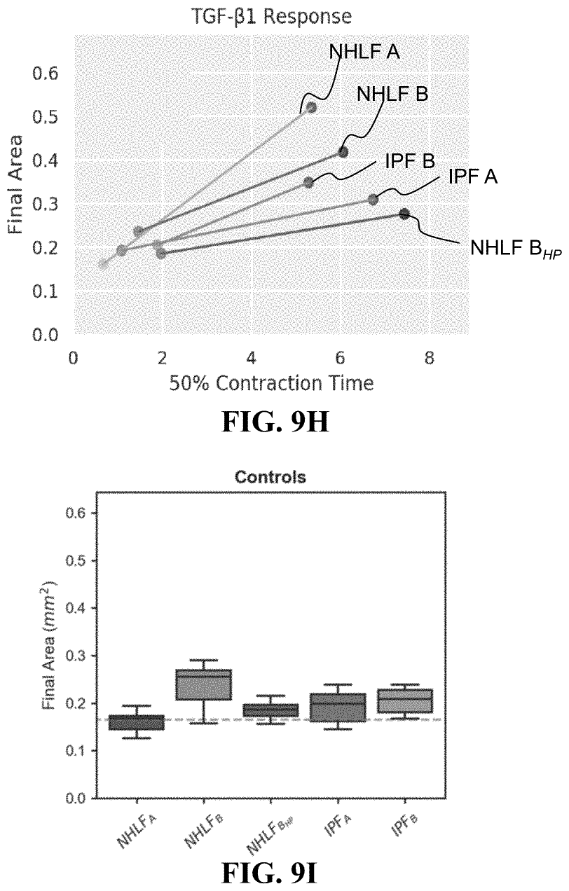

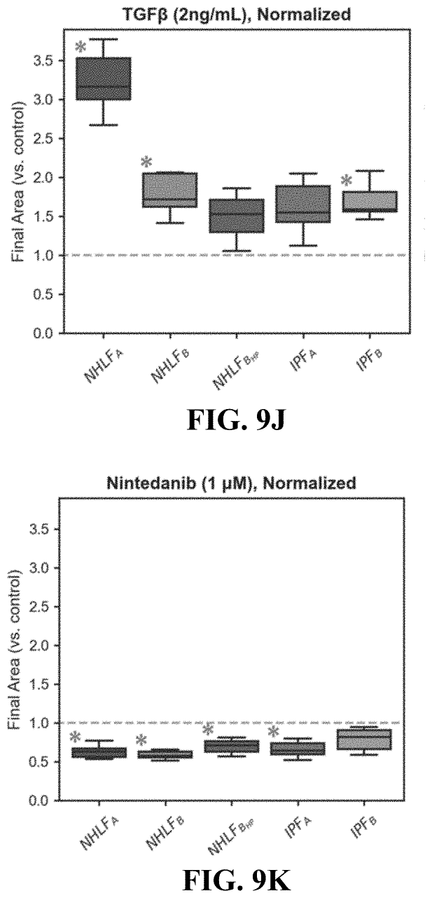

FIGS. 9A and 9B show histologic sections (color brightfield images are shown on the left with fluorescent images on the right) show final contracted assays for NHLF B and IPF B with picrosirius red staining with scale bars of 250 μm. FIGS. 9C through 9G provide individual plots for each fibroblast donor showing remodeling response to TGF-β1 and nintedanib (“Nint.”) stimulation. FIG. 9H provides TGF-β1 response compared between fibroblast donors with lines indicating the average responses with and without TGF-β1. Final areas are indicated in mm² and statistical differences are annotated on graphs in FIGS. 9I through 9K. FIG. 9I shows control conditions showed consistency in contraction with no significant differences in final contracted area. FIGS. 9J and 9K show final area normalized to each donor's mean control area to indicate fold-change in response to TGF-β1 (FIG. 9J) and nintedanib (FIG. 9K). (Statistical significance by two-way ANOVA P<0.05; Post-hoc Tukey test asterisk indicates P<0.05 compared to donor control; N=5 for all conditions).

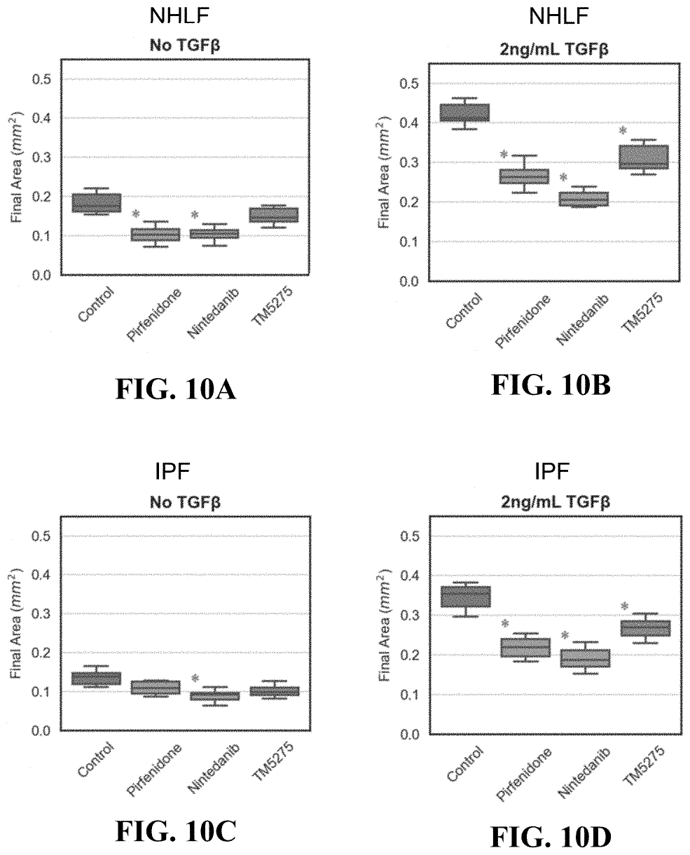

In FIGS. 10A and 10B, the IPF therapeutics pirfenidone, nintedanib, and TM5275 were evaluated on NHLF cells to determine the effects of these drugs on final assay area. In FIGS. 10C and 10D, the IPF therapeutics pirfenidone, nintedanib, and TM5275 were evaluated on IPF cells to determine the effects of these drugs on final assay area. Graphs for each cell type are separated into no TGF-β1 (a, c) and 2 ng/mL TGF-β1 (b, d). (Statistical significance by two-way ANOVA P<0.05; Post-hoc Tukey test *=P<0.05 compared to control; N=5 for all conditions).

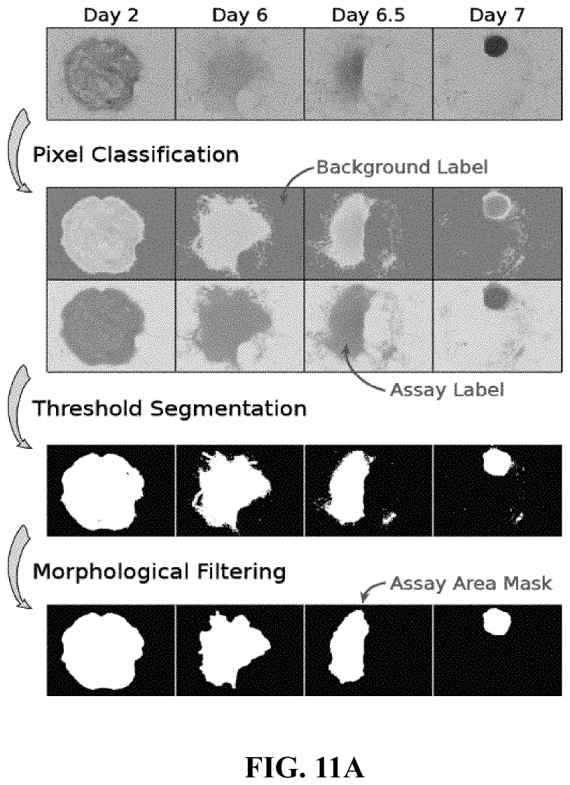

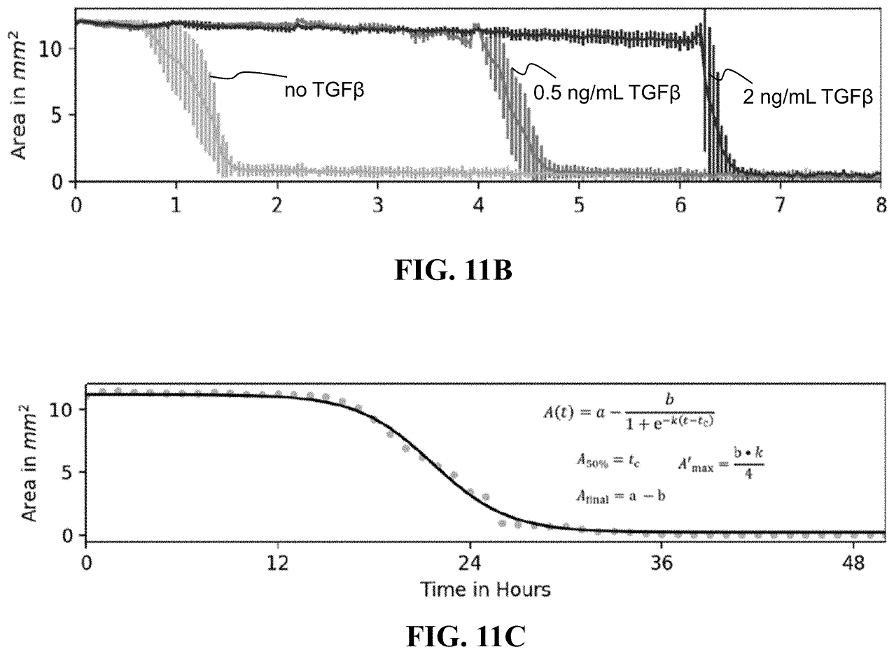

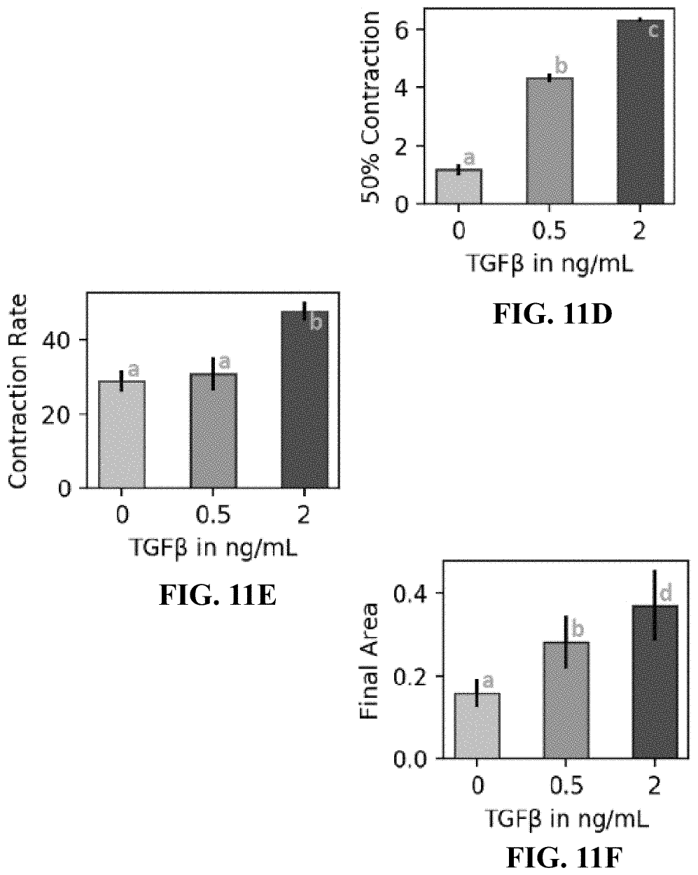

FIG. 11A shows an example segmentation approach utilizing Ilastik for pixel classification and the Python OpenCV library for thresholding and morphological filtering. Example images from diverse stages of assay remodeling were chosen to demonstrate the resilience of this segmentation approach to different image features. FIG. 11B shows the resulting masks enabled calculation of assay area, as illustrated with NHLF cells and different concentrations of TGF-β1. FIG. 11C shows an example measurement demonstrating image metric extraction for NHLF cells with no TGF-β1. For each individual microwell, the logistic function is fit using a least squares regression. This function enabled extraction of 50% contraction time (FIG. 11D), maximum contraction rate (FIG. 11E), and final area (FIG. 11F). Note that the 50% contraction time (vertical axis) is indicated here as days after start of assay, contraction rate in mm2 per day, and final area in mm2. (Statistical significance: ab, be=P<0.01; bd=P<0.05).

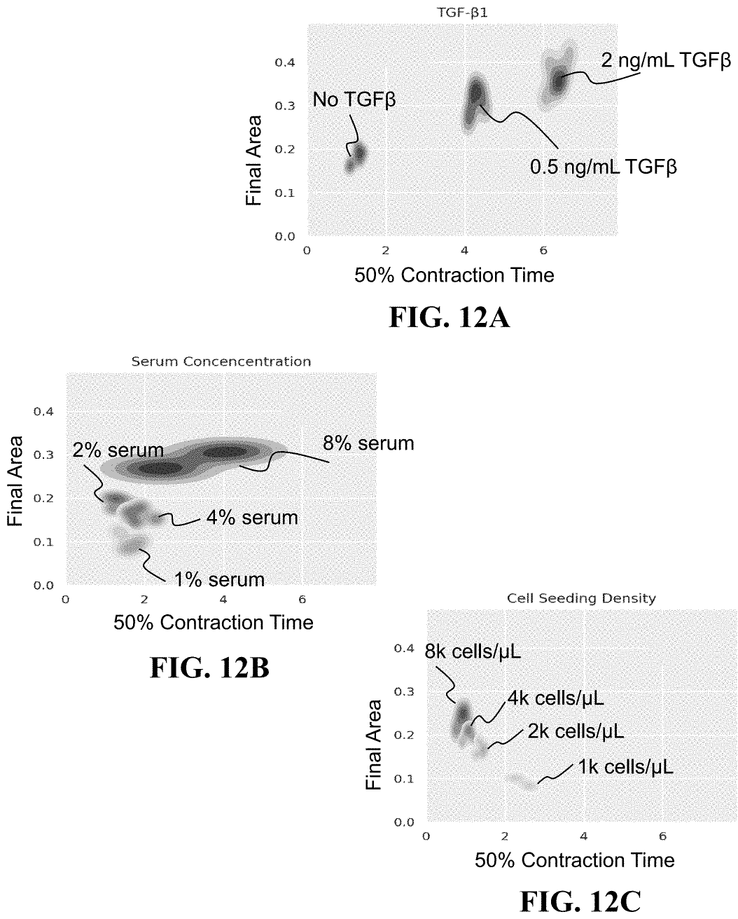

Output contraction times and final assay areas from image processing analysis were used to plot kernel density estimates showing interplay between contraction time and final assay area. FIG. 12A shows stimulation with TGF-β1 resulted in increases in both 50% contraction time and final assay area. FIG. 12B shows evaluation of serum concentration demonstrated relatively consistent contraction time with increasing final assay area in response to higher serum concentrations. FIG. 12C shows cell seeding density had an inverse relationship between contraction time and final assay area.

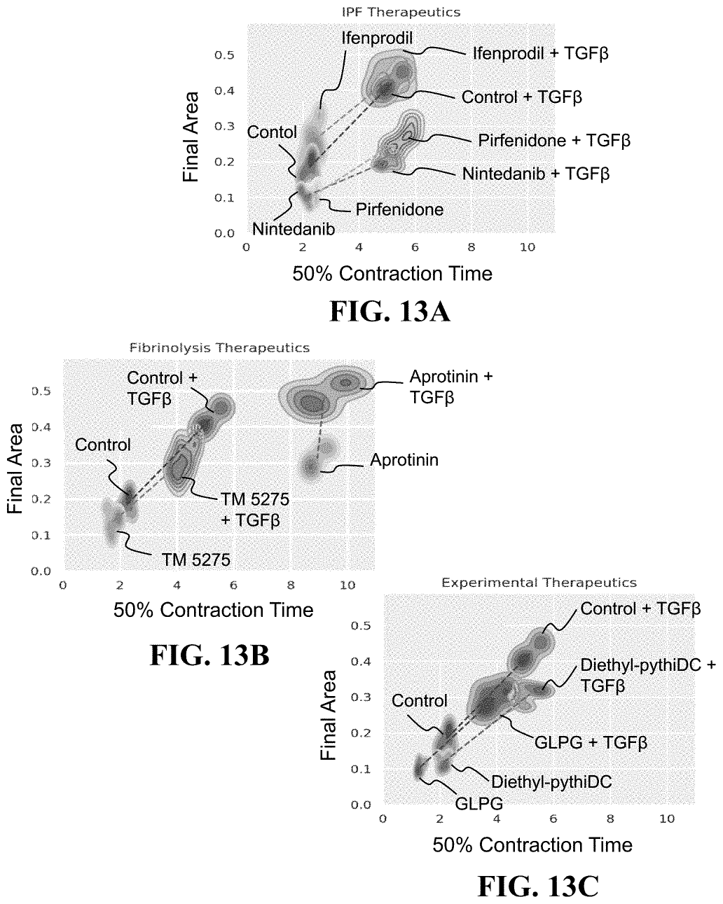

FIG. 13A shows time-course changes in assay area show the effects of IPF therapeutics on remodeling behavior. FIG. 13B provides evaluation with therapeutics targeting the fibrinolytic system demonstrate the impact of a PAI-1 inhibitor (TM 5275) and a tPA/uPA inhibitor (aprotinin). In FIG. 13C, additional experimental therapeutics were evaluated. In some embodiments, therapeutics can include ifenprodil, pirfenidone, nintedanib (“nint.”), aprotinin, TM 5275, diethyl-pythiDC, GLPG, and H₂O₂.

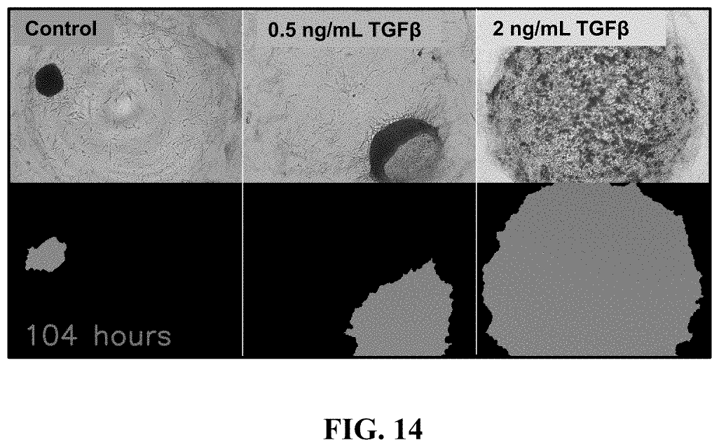

FIG. 14 shows ECM Remodeling, demonstrating altered remodeling of fibroblast laden fibrin scaffolds with different concentrations of TGF-β1. Higher concentrations result in delayed contraction and larger final size of the contracted matrix. Graphs below each micrograph demonstrate the image processing output of area masks for each time point. Micrographs were taken by the Incucyte S3 with 4× objective.

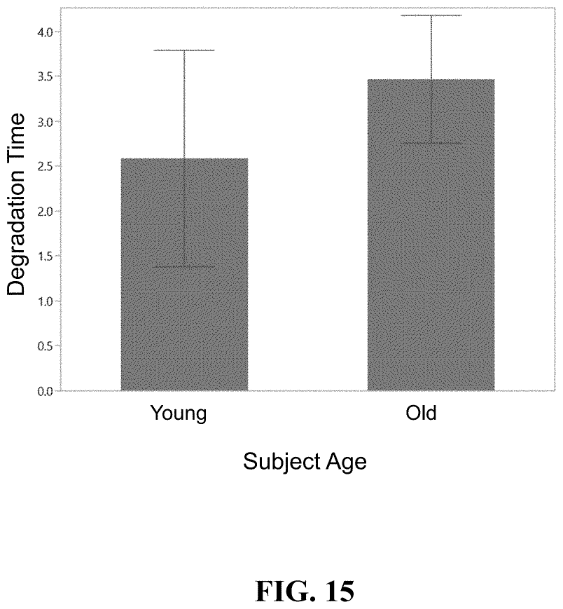

FIG. 15 provides a plot comparing fibrin degradation between different age animal subjects. In some embodiments, the method and system can be adapted to be used as diagnostic test. Using blood serum collected from young and old mice, significant differences in fibrin degradation rate can be detected between the different age mice. As shown in FIG. 15, slower fibrinolysis was detected to be significantly shower in older mice. In some embodiments, the system and method can be used as a diagnostic test for both age-related and lung diseases using a subject's serum samples, for example, in a fibrosis assay.

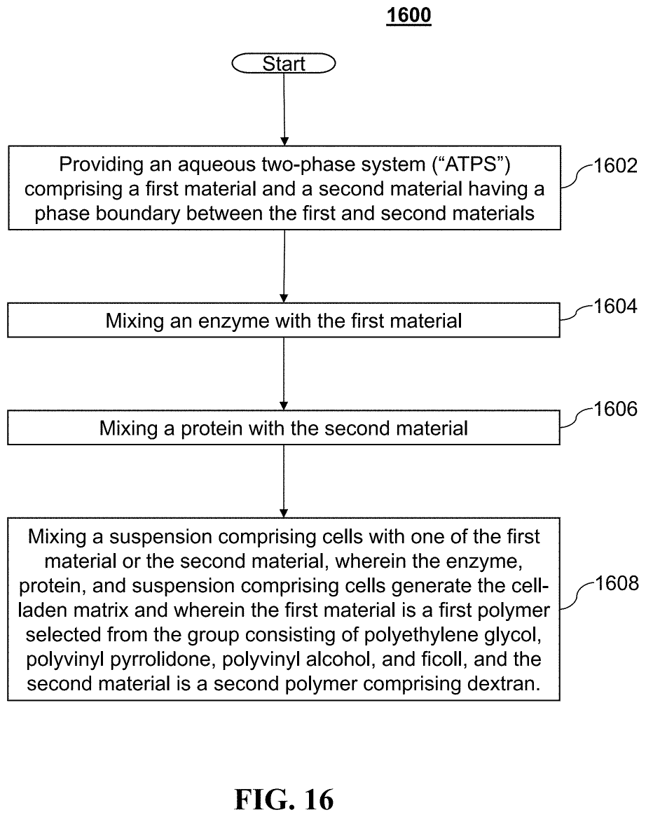

FIG. 16 provides an exemplary method 1600 for forming a microscale cell-laden matrix. Method 1600 can include a step 1602 of providing an aqueous two-phase system (“ATPS”) comprising a first material and a second material having a phase boundary between the first and second materials. At step 1604, method 1600 can include mixing an enzyme with the first material of the ATPS. At step 1606, method 1600 can include mixing a protein with the second material of the ATPS. At step 1608, method 1600 can include mixing a suspension comprising cells with one of the first material or the second material, wherein the enzyme, protein, and suspension comprising cells generate the cell-laden matrix and wherein the first material is selected from the group consisting of polyethylene glycol, polyvinyl pyrrolidone, polyvinyl alcohol, and ficoll, and the second material is dextran. Method 1600 can stop after step 1608 or can optionally include mixing the ATPS mixture with a third material comprising one or more additives. Method 1600 can stop after mixing the third material or can optionally include imaging the cell-laden matrix and the one or more additives.

In any of the embodiments disclosed herein, the system and method can be used for diagnosing diseases associated with alterations in fibrinolysis and/or fibrin remodeling including cellular deposition of novel ECM material and subsequent contraction of the material. For example, a body fluid sample from a patient with such a disease can be used in combination with a system and/or method disclosed herein.

In any of the embodiments disclosed herein, the system and method can be used to identify potential therapeutic compounds that alter fibrinolysis and/or fibrin remodeling, including high-throughput analyses of such therapeutic compounds. High throughput multi-well analyses can be adapted for use with a method or system disclosed herein for diagnostic and/or drug discovery applications.

In any of the embodiments disclosed herein, the system and method can be used in combination with one or more imaging methods for high throughout multi-well analyses.

The following examples further illustrate aspects of the present disclosure. However, they are in no way a limitation of the teachings or disclosure of the present disclosure as set forth herein.

### Examples

### Example 1: Fibrin Assay Cell Culture and ATPS Reagents

A stock solution of DEX (20% w/w dextran T500; Sigma) was prepared in phosphate buffered saline (PBS) on a rocker overnight. A stock solution of PEG (6% w/w, 35 k MW; Sigma) was prepared in fully supplemented culture media with 10% deionized water to balance osmolality. Both stock solutions were passed through a 0.22 um sterilizing syringe filter before storage. PEG working solutions were stored for up to 2 weeks at 4° C. Thrombin (Human Alpha Thrombin; Enzyme Research Labs) was also added to the PEG solution at a concentration of 0.1 U/mL immediately preceding experiments. Fibrinogen-DEX solutions were prepared by diluting fibrinogen stock solution (human fibrinogen 3; Enzyme Research Labs) to a final concentration of 4 mg/mL in a sterile solution of 4% 10×DMEM, 15% DEX stock solution (to a final concentration of 3% dextran), and 50% cell suspension in growth media. For all experiments excluding cell concentration evaluation, the cell suspension was diluted for 1000 cells per microliter in the final fibrinogen-DEX solution.

### Example 2: Cell Preparation

Normal human lung fibroblasts (NHLF lot #0000580583; Lonza) from a 79-year-old female with a history of smoking, and idiopathic pulmonary fibrosis fibroblasts (IPF lot #0000627840; Lonza) from a 52-year-old male were cultured in fibroblast growth media (FGM; Lonza). Cells were passaged at 80-90% confluence and were sub-cultured in 1:3 ratios by trypsinization. When at the desired confluence, cells were washed with PBS and 0.05% trypsin solution was added to the flask. Cells were incubated for 2 min, diluted with fibroblast growth media, and then harvested and centrifuged (200× g, 5 min) in a conical tube. The supernatant was aspirated and the cell pellet was re-suspended in serum-free culture media. When used in fibrin degradation experiments, cells were re-suspended at 2× the final desired concentration (1000 cells/ul unless otherwise indicated). All experiments were conducted with cells at or below passage 12. In all experiments, media was changed every 48 hours and any media additives (plasminogen, TGF-β1, drugs, etc.) were included.

### Example 3: ATPS Printing of Fibrin Microgels

Working solutions of PEG with 0.1 U/mL of thrombin were warmed to 37° C. and pipetted into a 96-well plate. For production of droplets, fibrinogen-DEX solutions with cell suspension were maintained at 37° C. and pipetted directly into the PEG-thrombin media using either a manual pipette or a semi-automated 96-channel pipette (Viaflo-96; Integra). All assays utilized a volume of 1 μl unless otherwise noted. Following dispensing of the DEX phase, the plates were placed in an incubator at 37° C. for 30 min to allow the thrombin to enzymatically crosslink the fibrinogen into a fibrin matrix (FIG. 1A). The PEG-enriched media was then washed four times by removing, then replacing half of the media with PEG-free media. When applicable, the final media addition was supplemented with stimuli as detailed in Example 5 below. For the duration of each experiment, assay plates were imaged every 2 hours at 4× with an automated cell culture monitoring system (Incucyte S3; Essen Biosystems). After one day of culture, plasminogen (50 μg/mL) (Human Glu-Plasminogen; Enzyme Research Labs) was added as a 10× concentrated solution to each well in order to initiate assay degradation (FIG. 1B), unless otherwise noted for specific conditions. Fresh plasminogen was included with each subsequent media addition. Positive controls with active plasmin (1 U/ml) (Human Plasmin; Enzyme Research Labs) and negative controls without plasminogen were included in each experiment. As cells activated plasminogen, the fibrin scaffold progressively degraded as illustrated in FIG. 1C.

In order to print fibrin into letters and arbitrary shapes, a 6% PEG solution containing 0.1 U/ml of thrombin was pipetted into a 6-well plate and warmed to 37° C. A 6% DEX solution containing 8 mg/mL fibrinogen was pipetted directly into the PEG phase to manually draw the desired shapes. After 30 min, darkfield images were taken on a stereoscope (Leica S6 E) to visualize the printed fibrin scaffold.

For cell viability measurements, 1 μl fibrin scaffolds were printed each containing 5000 cells total. The fibroblast-laden scaffolds were maintained in serum free media for 24 hours before live/dead staining (ReadyProbes™ Cell Viability Imaging Kit; Invitrogen). NucBlue and NucGreen (staining for total cells and dead cells respectively) were applied according to manufacturer directions. Dead control scaffolds were treated with 70% ethanol for 15 min prior to staining. Scaffolds were fluorescently imaged to assess viability. For calculation of percent viability, the density counting workflow in ilastik was used to count the number of total cells and dead cells.

### Example 4: High-Throughput Brightfield Image Analysis

After each experiment, brightfield images for every time point were downloaded in jpeg format from the automated cell culture monitoring system. Python's OpenCV library was implemented for the masking approach illustrated in FIG. 2A. First, a threshold was set at 50% of the maximum intensity (128 for 8-bit integer pixel values) in order to isolate the darker pixels of semi-opaque fibrin hydrogel from the background of the image. A closing morphological filter with a 25×25 kernel was then applied to each mask in order to remove noise. This masking approach was applied to the initial time point from every experimental condition in order to establish the relevant assay area for downstream measurements. As fibrin degrades during an experiment, the average pixel intensity within the masked area increases accordingly (FIG. 2B). The automated live-cell imager (Incucyte S3; Essen Biosystems) automatically adjusts brightness to maintain consistent white balance between images. For experiments involving multiple assay volumes, image brightness was scaled to maintain consistent background intensity.

For each experimental replicate, a sigmoid curve was fit using the curve fit function from the SciPy library in Python. The logistic function given by the equation in FIG. 2C enabled automated extraction of the time point for 50% degradation, as well as the maximum slope at the equation's centroid (FIGS. 2D and 2E).

### Example 5: Phenotypic Evaluation of Stimuli

In order to evaluate fibrin degradation rate with a known anti-fibrinolytic stimulus, various concentrations of transforming growth factor type 131 (Human Recombinant TGF-β1; Peprotech) were added to the assay media after ATPS polymers were rinsed out of the microplates.

To evaluate the capability of this assay to test the fibrinolytic effects of therapeutic stimuli, a variety of drug compounds were introduced to the fibrinolysis assays after the wash step. This included 400 μM pirfenidone (Selleck Chem), 0.4 μM nintedanib (Selleck Chem), 100 μM hydrogen peroxide (Sigma), and 20 μM diethyl-pythiDC (AOBIOUS). These concentrations were established in preliminary experiments that evaluated a range of concentrations used in prior literature. Stimuli were freshly mixed for each media change during experiments, and a minimum of four replicates were tested per experimental condition.

### Example 6: Statistical Analysis

All experimental values are reported as means±standard deviation. ANOVA tests were performed using the statsmodels library in Python 3 with the Tukey test for post-hoc pairwise comparisons.

### Example 7: Fabrication of Microscale Fibrin Scaffolds

The development and characterization of the cell-mediated fibrinolysis assay was focused on establishing a microplate-compatible fibroblast-laden fibrin scaffold and verifying the ability to distinguish between subtly different fibrinolytic environments. First, an ATPS approach was implemented to enable accurate printing of unprecedentedly small cell-laden fibrin scaffolds. Then, an automated image processing approach quantified fibrin degradation data from label-free brightfield images. Next, the established fibrinolytic effects of cell density and TGF-β1 were used to validate the assay's capability to distinguish between conditions. Finally, the microscale cell mediated fibrinolysis assay was implemented to evaluate the effects of anti-fibrotic therapeutics on fibroblasts from normal and diseased donors.

Biological environments establish fibrin matrices through coagulation, where a cascade of clotting factors activates thrombin, which enzymatically crosslinks fibrinogen into fibrin. Similarly, in vitro fibrin scaffolds are formed by exposing monomeric fibrinogen to thrombin. Fibrin has been used extensively in a wide variety of tissue engineering applications, but it is generally implemented as a bulk cast hydrogel. The conventional bulk casting procedure mixes thrombin and fibrinogen solutions by micropipette; however, this method cannot consistently handle small volumes (under 100 μl) due to adhesion of the partially coagulated mixture to pipette tips.

There have been a few applications of fibrin bio-printing that control crosslinking by alternating between layers of fibrinogen and thrombin, but this poses limitations to accuracy and reproducibility due to lack of control over fibrinogen's exposure to thrombin. There have also been a variety of applications for fibrin microbeads where oil immersions were used to disperse microbeads during crosslinking in oil-suspended droplets, but this results in inconsistent size and cells must be added separately after the microbeads have been washed. Reliable microscale volume and microplate compatibility were necessary to enable high-throughput adaptation in this assay. Precise control over cell seeding density was also vital for this approach due to its effect on remodeling rate.

A new approach to maintain fibrinogen in a distinct droplet and control diffusion of thrombin into fibrinogen during the polymerization process was established by implementing an ATPS with PEG and DEX. Above their critical concentrations, these soluble polymers thermo-dynamically drive aqueous systems to form two distinct phases. A previous ATPS microscale adaptation from related research for collagen contraction demonstrated consistency in response between the conventional 100 μL assay and ATPS microscale volumes. This work specifically took advantage of the short length scales for time-dependent and burst stimulation profiles, which would not be possible with conventional approaches due to diffusion constraints. A similar ATPS adaptation suited the approach described herein, and enabled fabrication of microscale fibrin scaffolds with standard liquid handling equipment to facilitate microplate compatibility.

During the initial optimization of PEG and DEX concentrations, lower concentrations were found to be unstable and resulted in fissure of the ATPS droplet. In order to maintain stable separation of phases during polymerization, minimum assay concentrations of 6% 35 kDa PEG and 3% T500 dextran were determined for stability during crosslinking (FIG. 1A). As a demonstration of the printing capabilities enabled by controlled enzymatic crosslinking, this formulation was pipetted into specific letters and shapes. Fibroblast viability has previously been verified at these ATPS concentrations in a prior microscale assay adaptation of collagen scaffolds. Fibroblast viability was evaluated for this fibrin ATPS procedure, which demonstrated 88.5±0.6% viability.

The necessity for this ATPS environment in the microscale fibrin degradation assay described herein comes from the capability of aqueous two-phase partitioning to control the timing of thrombin diffusion into the fibrinogen droplet. This control over timing restricts enzymatic crosslinking of cell-laden fibrin matrices until after the droplets have been dispensed (FIG. 1A). After a 30-minute incubation period, the fibrin was sufficiently polymerized and the ATPS solutions could be removed and replaced with growth media and stimulants for specific conditions (FIG. 1B). Bioengineered tissues were incubated for an additional period of 24 hours in regular growth media in order to allow cells to anchor themselves to the fibrin matrix before adding exogenous plasminogen.

After plasminogen was added to the wells, various activators and inhibitors produced by cells regulate the conversion of plasminogen into plasmin. Control conditions for each experiment verified rapid matrix degradation with the addition of exogenous plasmin and no matrix degradation when plasminogen is omitted. As the assay proceeds, the fibrin matrix gradually degrades with activated plasmin cleaving fibrin into soluble fibrin degradation products (FIG. 1C). This is visually evident by the disappearance of the opaque fibrin matrix. The following section is focused on implementing an image processing and analysis approach that enabled automated quantification of differences in fibrin degradation between conditions.

### Example 8: Label-Free Quantification of Fibrin Degradation

Due to the relative opacity of the fibrin scaffolds, pixels within the assay area are significantly darker than those in the background of microscope images. This enabled an analysis approach based on pixel intensities within the assay area. Many established hemostasis assays take advantage of fibrin's attenuation of light for quantification. These assays generally implement plate readers to measure absorbance during coagulation and fibrinolysis. Evaluation of this assay in a microplate reader may therefore serve as an alternative to brightfield analysis. However, the approach favored evaluation of pixel intensity from brightfield images so that the micrographs could serve as validation of assay progression. Unfortunately, the commercial image analysis package embedded in the live cell imaging system was not able to reliably discern the microprinted fibrin scaffold. An alternative image analysis pipeline was developed using Python's OpenCV library.

In order to isolate the assay area from background, a thresholding approach was sufficient because of the significant difference in pixel brightness. Here, any pixels brighter than the specified threshold were classified as background. A closing morphological filter was applied to the thresholded images to remove noise left by the thresholding process. FIG. 2A demonstrates mask generation and its implementation at later time points as the fibrin matrix degrades. After isolation of the assay area, average pixel intensity within masked regions was plotted in order to visualize time-course fibrin degradation (FIG. 2B). Fitting time-course data from each individual well with a sigmoidal curve facilitated extraction of the time point for 50% matrix degradation, as well as the maximum slope at the sigmoid's centroid (FIG. 2C).

FIG. 2D shows changes in the 50% degradation time point in response to different plasminogen addition times. The 50% degradation time point is shown as days since plasminogen addition. Increases in bar height indicate slower cell-mediated fibrinolysis. ANOVA indicated statistical significance of these differences in degradation time (P<0.01), and post-hoc pairwise analysis with the Tukey test demonstrated statistically significant differences between specific conditions (FIG. 2D). The increase in time to 50% degradation for later plasminogen additions indicates significant changes in the scaffold or cells in the first 24 hours. It has previously been demonstrated that cell-matrix interactions influence the rate of fibroblast-mediated fibrinolysis, so additional time before plasminogen addition may have influenced rates observed here through similar pathways. Hence, it was important in subsequent studies to evaluate cell-mediated fibrin degradation with a consistent plasminogen addition time. A plasminogen addition time at 24 hours was implemented so that fibroblasts could initiate cell-matrix interactions. This 24-hour addition of plasminogen was chosen to allow cells to recover from trypsinization and minimize residual trypsin activity.

The effects of assay volume were also evaluated. Assay volumes between 0.5 μl and 8 μl could be consistently printed and viewed within the field-of-view of a 4× microscope objective (FIG. 3A). The Python-based image masking approach was compared against a manual approach that outlined the assay area in image J with no significant differences in cross sectional area between techniques (FIG. 3B). Cross sectional area was also compared to volume through evaluation of geometric models. Compared against spheres, hemispheres, and spherical caps; a doubled spherical cap fit the volume and area data most closely as determined through least squares regression (FIG. 3C).

In prior microscale adaptation of collagen contraction, different assay volumes were found to maintain consistent contraction rates as long as cell density was maintained. Fibrinolysis trends in the experiments described herein also depend on cell density rather than assay volume. While the pixel intensity of higher volume assays had lower starting values, this reflected the presence of more fibrin which resulted in decreased transmission of light through those assays (FIG. 3D). Time points for 50% degradation, as determined by a sigmoid fit, showed no significant difference in degradation timing between different volume conditions (FIG. 3E). This consistency in degradation timing indicates similar rates of cell-mediated fibrinolysis between different volume conditions. Differences in maximum slope between conditions followed the same trend as differences in initial pixel intensity, resulting from the decreased transmitted light through higher volume fibrin scaffolds.

The consistency in degradation rates between volume conditions indicates uniformity in fibrin organization. Fibrin network morphology has a significant impact on fibrinolysis rate, where tight fibrin conformations degrade at a slower rate than scaffolds with looser fibrin conformations and thicker fibers. This suggests that at the concentration of thrombin used in the present assays, ATPS-mediated control over the diffusion of thrombin into the fibrinogen-containing phase results in consistent fibrin organization across the range of assay volumes tested.

### Example 9: Effect of Cell Seeding Density and TGF-β1

Cell seeding density was also evaluated. Conditions with higher seeding densities demonstrated significantly faster fibrinolysis (FIG. 4), with decreased time points for 50% degradation and increased maximum slope (FIGS. 4B and 4C; P<0.05 for all pairwise comparisons). The linear relationship between rate of fibrinolysis and cell number is consistent with a cell-mediated step being rate limiting in this process. This also highlights the importance of consistent cell-seeding density in fibrin printing applications. The ATPS printing technique is uniquely capable of establishing microscale cell-laden fibrin scaffolds with a consistent seeding density. However, seeding densities higher than 5000 cells per microliter could not be consistently established due to an increased viscosity that interfered with pipetting.

TGF-β1 is an established pro-fibrotic stimulus with well-characterized anti-fibrinolytic effects. Various concentrations of TGF-β1 were used to stimulate NHLF cells in the fibrin assays (FIG. 4D). Increasing concentrations resulted in longer time delays before fibrinolysis. The time points for 50% degradation further demonstrate this trend (FIG. 4E). All pairwise differences in 50% degradation time between conditions were significant with P<0.05 (FIG. 4F). Interestingly, these differences in fibrinolysis profile appear as a delay before initiation of fibrin degradation. Prior studies have linked elevated PAI-1 with delayed fibrinolysis, and TGF-β1 stimulation is closely associated with upregulation of PAI-1. However, TGF-β1 is also involved in fibroblast proliferation and matrix production, so a variety of factors are likely involved in the altered fibrin degradation.

In NHLF cells, a significant effect of cell passage number on fibrinolysis was also noticed. Higher passage numbers exhibited progressively longer 50% degradation times with slower fibrin degradation rates. These incidental observations are consistent with prior studies which demonstrate inhibition of fibrinolysis in senescent fibroblasts in vivo and in vitro due in part to the upregulation of PAI-1.

### Example 10: Evaluation of Hydrogen Peroxide, Therapeutics and IPF Fibroblasts

Having established baseline cell response measurements for fibrinolysis of the bioprinted fibrin micro-scaffolds, fibrinolytic profiles were compared between normal and diseased lung fibroblasts with a number of stimulants and inhibitors. Hydrogen peroxide is a reactive oxygen species (ROS) known to be produced by cells in response to TGF-β1 stimulation, while nintedanib and pirfenidone are the only two FDA-approved therapies for IPF. Diethyl-pythiDC, an experimental anti-fibrotic drug, is an inhibitor of certain prolyl 4-hydroxylases that play a role in post-translational modification of collagen and other proteins. The plasmin control condition was included in graphs for reference but was excluded from statistical analysis in the interest of focusing on therapeutic conditions of interest.

A general comparison between normal and diseased fibroblasts (FIG. 5A-5H) demonstrates that cells from the IPF donor consistently degraded fibrin significantly faster than the normal fibroblasts (P<0.01 by two-way ANOVA). However, prior research indicates that diseased fibroblasts from IPF donors express elevated levels of PAI-1 and should consequently exhibit slower fibrin degradation. This unexpected decrease in fibrinolysis time in IPF fibroblasts may be due to the cells' extended removal from the diseased microenvironment. In the diseased lung, overactive epithelial cells secrete several growth factors, cytokines, and chemokines involved in migration, proliferation, and activation of fibroblasts. Additionally, the donor for these NHLF cells does not fit the typical profile for healthy lung tissue. This particular donor was a 79-year-old female with a history of smoking. Age related cellular senescence and tobacco use have both been associated with increased levels of PAI-1, so the fibrinolytic system in these “normal” fibroblasts may be dysregulated compared to a younger non-smoking donor.

Stimulation with hydrogen peroxide (H₂O₂) alone demonstrated highly significant decreases in the rates of fibrinolysis (P<0.1) suggesting a critical role of ROS in the process of cell-mediated fibrinolysis. In contrast, conditions that included TGF-β1 showed no significant difference upon further stimulation with exogenous hydrogen peroxide. This non-additive effect is consistent with a notion that the effects of adding exogenous H₂O₂ and exogenous TGF-β1 converge. That is, TGF-β1-triggered increase in endogenous H₂O₂ production, may mask effects of any exogenous H₂O₂ addition. Such effects may also work in concert with ROS-induced reduction in TGF-β1 receptors.

The two FDA-approved IPF drugs, nintedanib and pirfenidone, did not show a significant impact on fibrinolysis. These therapeutics have established anti-fibrotic effects, so these results indicate that the mechanism of action for nintedanib and pirfenidone has little relation to fibrinolytic activity of lung fibroblasts. Given the limitation that nintedanib and pirfenidone can slow but not stop or reverse IPF, the ability to test IPF-relevant pathways such as fibroblast-mediated fibrinolysis-associated processes that these drugs do not target may provide opportunities for developing co-therapeutics or alternatives with enhanced efficacy. The experimental drug diethyl-pythiDC significantly (P<0.05) delayed fibrinolysis. Diethyl-pythiDC is a selective inhibitor of prolyl 4-hydroxylase, an enzyme best known for structure-stabilizing modifications of collagen that also acts on a variety of proteins including hypoxia inducible factor 1. The ability of diethyl-PythiDC to reduce fibroblast-mediated fibrinolysis is a novel finding and demonstrates the utility of the assay described herein. Given the many physiological factors present in blood or expressed by many types of cells positively (e.g. proteases such as uPA, tPA, cathepsins, FXIa, FXIIa, kallikreins) and negatively (e.g. serpins such as PAI-1 and α2-antiplasmin, α2-macroglobulin) impact fibrinolysis, it is possible that this fibrin printing and fibrinolysis assay will be of broad utility.

This work describes an approach for ATPS-based printing of microscale cell-laden fibrin scaffolds. A droplet comprised of the heavier phase partitions cells and fibrinogen while the bulk phase provides thrombin to promote localized enzymatic crosslinking, leading to controlled production of microliter-scale fibrin constructs. Automated label-free image processing quantified rates of cell-mediated fibrin degradation from time-course brightfield images. Primary human lung fibroblasts were found to degrade the fibrin scaffold at a rate dependent on source of cells, cell density, and the presence of soluble factors. Given the variety of contributors to dysregulation of fibrinolysis seen in cancer, fibrosis, and metabolic disease; this phenotypic assay for cell-mediated fibrin degradation provides a potentially valuable research tool for further studies in these and other fields. Additionally, the technique developed here for aqueous two-phase printing of cell-laden fibrin by in situ enzymatic cross-linking can be broadly applied in bio-printing and tissue engineering applications.

### Example 12: Collagen Spheres from Fibrin Drop Cell Preparations

Human primary lung fibroblasts were used in all experiments presented in this paper. Unless otherwise noted, experiments utilized normal human lung fibroblasts (NHLF B lot #0000580583; Lonza) from a 79-year-old female with a history of smoking. For experiments evaluating donor variability, the following cells were utilized: NHLF A (NHLF lot #0000608197; Lonza) from a 67-year-old male, IPF A (IPF lot #0000627840; Lonza) from a 52-year-old male, and IPF B (IPF lot #6F5002; Lonza) from an 83-year-old male. All cells were cultured in fibroblast growth media (FGM; Lonza). Cells were passaged at 80-90% confluence and were sub-cultured in 1:3 ratios by trypsinization. When at the desired confluence, cells were washed with PBS and 0.05% trypsin solution was added to the flask. Cells were incubated for 2 min, and then harvested and centrifuged (200 μg, 5 min) in a conical tube. The supernatant was aspirated and the cell pellet was re-suspended in FBS-free culture media. When used in fibrin degradation experiments, cells were re-suspended at 2× the final desired concentration (2500 cells/ul unless otherwise indicated). To promote collagen production and contraction in addition to fibrin degradation, serum (bovine or human) were added at 2% or higher up to about 10%. Higher cell densities (about 2500 cells/ul and higher up to about 10,000 cells/ul) also promote collagen production. All experiments were conducted with cells at or below passage 8 except for high passage experiments conducted at passage 12. In all experiments, media was changed every 48 hours and any media additives (plasminogen, TGF-β1, drugs, etc.,) were included.

### Example 13: ATPS Printing of Fibrin Microgels

ATPS printing of fibrin micro-scaffolds has previously described above. Briefly, working solutions of PEG with 0.1 U/mL of thrombin were warmed to 37° C. and pipetted into a 96-well plate. For production of droplets, fibrinogen-DEX solutions with cell suspension were maintained at 37° C. and 4 μl per assay (unless otherwise noted) was pipetted directly into the PEG-thrombin media using a semi-automated repeater pipette (Repeater E3X; Eppendorf). Following dispensing of the DEX phase, the plates were placed in an ambient air incubator at 37° C. for 30 min to allow the thrombin to enzymatically crosslink the fibrinogen into a fibrin matrix (FIG. 6A). The PEG-enriched media was removed using a 12-channel micropipette and replaced with 100 μl of fully supplemented media in each well. When applicable, this media addition was supplemented with stimuli as detailed in example 17 below. For the duration of each experiment, assay plates were imaged every 2 hours at 4× with an automated cell culture monitoring system (Incucyte S3; Sartorius). As the assay proceeded, the fibroblasts progressively remodeled the fibrin scaffold as illustrated in FIG. 6C.

### Example 14: Histologic Analysis of Fibrin Microgels

Contracted cell-ECM spheroids were harvested after 9-12 days of culture. These structures were prepared for histology, stained, and imaged as previously described for cultured spheroids. Briefly, the spheroids were washed with PBS and fixed in 4% paraformaldehyde (Alfa Aesar) for 1 hour at room temperature. The structures were stained with 0.5% methylene blue solution in PBS for 10 minutes at room temperature to aid in visualization during histology. Samples were placed in a cryomold containing optimal cutting temperature (OCT) compound, and flash frozen in cooled isopentane. 10 um sections were obtained using a CryoStar NX70 cryostat (Thermo Fisher Scientific).

Upon warming to room temperature, the sections were washed with PBS, permeabilized with 0.2% Triton-X 100, and blocked for 1 hour at room temperature with 4% bovine serum albumin (BSA) (Millipore Sigma). Sections were stained for 30 min at room temperature with Sirius red (0.1% of Sirius red in saturated aqueous picric acid), as previously described for collagen bundle staining. The samples were then washed with PBS, stained with DAPI for 15 minutes at room temperature, and coverslipped. Samples were imaged using a DMi8 microscope (Leica) equipped with 10× and 20× air objectives. Fluorescence was detected using Texas Red channel settings as previously described. Mean fluorescence intensity was quantified in ImageJ as the average pixel intensity within the sections.

### Example 15: mRNA Quantification by qPCR

RNA extraction and qPCR: Cells from 12 wells at the indicated time points were pooled together per condition and lysed with 350 μl of RLT lysis buffer. RNA was extracted using a RNeasy Mini Kit (Qiagen, #74104) and was performed according to the manufacturer's instructions. RNA sample concentration was measured using a NanoDrop OneC Spectrophotometer (Thermo Fisher Scientific). A High-Capacity RNA-to-cDNA Kit (Applied Biosystems, #4387406) was used for reverse transcription; 400 ng of RNA for each sample was mixed with 10 μl primer, 1 μl reverse transcriptase enzyme and nuclease-free water to bring the final reaction volume to 20 μl. The reaction was performed for 60 minutes at 37° C., followed by 5 minutes at 95° C. using a Veriti Thermal Cycler (Applied Biosystems). qPCR was performed using a QuantStudio 3 Real-Time PCR System (Applied Biosystems). Each reaction consisted of 1 μl cDNA, 10 μl TaqMan Fast advanced master mix (Applied Biosystems, #4444556), 1 μl primer, and 6 μl nuclease-free water. TaqMan primers (Applied Biosystems) for smooth muscle actin (ACTA2, Hs00426835_g1), plasminogen activator (PLAT, Hs00263492_m1), Plasminogen activator inhibitor-1 (SERPINE1, Hs00167155_m1), collagen type I (COL1A1, Hs00164004_m1), plasminogen activator (PLAU, Hs01547054_m1), and Ki67 (MKI67, Hs01032443_m1) were utilized. The QuantStudio 3 was programmed with a 2-minute hold at 95° C., followed by 40 cycles of 95° C. for 1 second and 60° C. for 20 seconds. Each sample was run with biological triplicates. The relative gene expression was calculated using the 2-AACT method, with glyceraldehyde-3-phosphate dehydrogenase as the housekeeping gene (GAPDH, Hs02786624_g1). Fold changes were normalized with respect to the time zero timepoint with no TGF-β1 stimulation and are reported as the mean with the error bars representing the minimum and maximum values.

### Example 16: Brightfield Determination of Final Area

After each experiment, the final projected areas of the cell-ECM spheroids were determined from brightfield images taken at the final time point using a benchtop imaging system (2× objective; EVOS M7000; ThermoFisher). These images were then segmented through a process of pixel classification, thresholding, and morphological filtering in order to isolate the cell-ECM construct area from the background.

Pixel classification implemented Ilastic, a freely available image classification tool developed by the European Molecular Biology Laboratory. Ilastik's pixel classification utility implements a random forest classifier for quick and robust segmentation. In order to train the classifier, 10 characteristic images were selected to include different stages of ECM remodeling. In this step, each individual pixel is assigned a probability for belonging to layers for the background or the cell-ECM construct. Ilastik enables interactive training of the random forest classifier via user annotations of the training images. All default features (G=0.3 through 10 for intensity, edge, and texture) were utilized for this interactive training by methodically annotating mislabeled areas of each training image. Care was taken to equally annotate background and cell-ECM areas in order to prevent the algorithm from weighting features inappropriately. Through this iterative training method, the user can evaluate interactive predictions by the algorithm and then draw additional annotations to correct mistakes. When additional training annotations no longer improved background noise and edge feature fit of the predicted mask over the cell-ECM area, the trained classifier was saved for future use. With each experiment, this trained classifier was reloaded and classification performance was evaluated on representative images (not from the training set) before use.

This pixel classification workflow performs semantic segmentation, and therefore returns a probability map for the background and cell-ECM area for each image. The probability map was transformed into background and cell-ECM area objects through thresholding. Thresholding of these probability masks then enabled generation of a single mask to isolate the cell-ECM area. A closing morphological filter with a 25×25 kernel was then applied to each mask in order to remove noise. The area from segmented masks was then used to quantify cell-ECM contraction.

Various concentrations of transforming growth factor type β1 (Human Recombinant TGF-β1; Peprotech) were used for validation due to its established anti-fibrinolytic and pro-fibrotic qualities. TGF-β1 was added at indicated concentrations in the assay media which was used to rinse and remove ATPS polymers after incubation.

### Example 17: Phenotypic Evaluation of Stimuli

As described above in example 12, the customized high-throughput image analysis approach can also be applied for phenotypic evaluation of all experiments. In order to evaluate ECM remodeling behavior with established stimuli; experiments implemented different conditions of TGF-β1, FBS, and cell seeding density. TGF-β1 was introduced at concentrations of 0, 0.5, 2, and 10 ng/mL; however the highest concentration did not contract within the duration of the experiment and was therefore omitted from analysis. In order to evaluate the remodeling effects of serum, fetal bovine serum (FBS, Lonza) at concentrations of 0, 1, 2, 4, and 8% by volume of the cell culture media was added during the washing step after fibrin crosslinking. For cell seeding density experiments, fibroblasts were suspended at appropriately modified concentrations in the dextran phase of the ATPS fibrin printing formulation so that assays were printed with concentrations of 1, 2, 4, and 8 thousand cells per microliter within a 4 μl assay.

In order to evaluate the capability of this assay to test the fibrinolytic and anti-fibrotic effects of therapeutic stimuli, a variety of drug compounds were introduced to the assays after the wash step. This included 10 μM TM5275 (MedChemExpress), 1 μM nintedanib (Selleck Chem), and 500 μM pirfenidone (Selleck Chem); all diluted and stored according to supplier data sheet recommendations. These concentrations were established in preliminary experiments that evaluated a range of concentrations used in prior literature. These stimuli were freshly mixed for each media change during experiments, and a minimum of four replicates were tested per experimental condition.

### Example 18: Statistical Analysis

All experimental values are reported as means±standard deviation. ANOVA tests were performed using the statsmodels library in Python 3 with the Tukey test for post-hoc pairwise comparisons. Experiments involving two independent variables (such as therapeutic stimulus and TGF-β1) implemented two-way ANOVA to evaluate the significance of combined effects.

### Example 19: Fabrication of Microscale Fibrin Scaffolds

In tissue repair, fibrin formation is followed by fibroblast migration, fibrinolysis and matrix remodeling. In lung fibrosis, fibrinolytic activity is decreased and undegraded fibrin is commonly reported in human IPF patient lungs. The method to print microscale cell-laden fibrin gels to quantify cell-mediated fibrinolysis is described above in examples 1 through 11. The study conditions described above, however, do not provide readouts of fibroplasia such as collagen deposition. Furthermore, the fibrinolysis assay did not show significant change in response to treatment with fibrosis drugs such as nintedanib and pirfenidone.

Published larger-scale fibrin-based fibroplasia assays were used for skin keloids. Specifically, the plasminogen addition step was replaced with addition of FBS and TGF-β1 instead. Under these revised conditions, fibrin was gradually replaced by a collagen-rich ECM. Surprisingly, after 3-7 days, contraction and detachment of the remodeled cell-laden matrix into a compact cell-ECM spheroid can be observed. While early contraction of the fibrin gel itself has been reported and inhibition of collagen gel contraction by incorporation of fibrin has been reported, there are no reports of detachment and contraction of the ECM produced by cells embedded in fibrin gels. Here, the method tests whether this unexpected contraction, detachment, and spheroid formation process could be utilized as a convenient, image-based, direct readout of fibroplasia in fibrin droplet assays.

Biological environments establish fibrin matrices through coagulation, where a cascade of clotting factors activates thrombin to enzymatically convert fibrinogen into fibrin. Similarly, synthetic fibrin scaffolds are formed by exposing monomeric fibrinogen to activated thrombin. In the technique described above for generating fibrin micro-scaffolds utilized an ATPS with PEG and dextran to improve control over fibrin formation, which enables printing of unprecedentedly small cell-laden fibrin matrices with standard liquid handling equipment. As described above, fibroblast-mediated fibrinolysis can be initiated by addition of exogenous plasminogen comparable to levels found in serum. Here, decreasing availability of plasminogen to levels that may better reflect tissue levels by supplementing the media with FBS. These adjustments significantly altered the trajectory of the microscale fibrin remodeling process compared to the fibrin forming approach and to other published fibrin fibroplasia assays. Rather than strictly degrading the scaffold into fibrin degradation products and dissociated cells, these conditions induced deposition of significant amounts of collagen followed by contraction of the cell-ECM construct into a fibrotic spheroid.

In this approach, the microscale format enabled microwell plate implementations with convenient automated live imaging. In order to fit the printed microscale fibrin drops within the field of view of a 4× objective, ATPS printing was implemented as described above. The ATPS-based bioinks allowed partitioning of fibrinogen into the denser, DEX-rich, droplet phase while thrombin was allowed to diffuse in gradually from the less dense, PEG-rich, bulk phase solution. This controlled mixing of enzyme with fibrinogen delayed crosslinking of cell-laden fibrin matrices until after the fibrinogen droplets were dispensed (FIG. 6A). After a 30-minute incubation period, the fibrin was sufficiently polymerized and the ATPS solutions could be removed and replaced with growth media.

During assay progression, remodeling is visually apparent in brightfield images as opaque fibrin transitioning into a translucent fibrous matrix and eventually contracting into a dense spheroid (FIGS. 6B and 6C). This concurrent fibrinolysis and deposition of cell-secreted ECM is similar to in-fibrin fibroplasia processes reported previously although the final contraction into a dense spheroid is novel. In the absence of FBS, which contains plasminogen, the initial fibrin matrix remained opaque and intact with minimal change. Control conditions verified that presence of both FBS and cells was necessary for degradation of the opaque fibrin scaffold, indicating that cell mediated activation of plasminogen was necessary for fibrin degradation. Factors contributing to altered fibrinolysis, increased ECM deposition, and cell contraction are assessed in the following section.

### Example 20: Response to TGF-β1

Downstream signaling effects of TGF-β1 include inhibition of fibrinolysis, increased fibroblast activation, increased synthesis and deposition of ECM, inhibition of ECM breakdown, and increased contractility. This section describes how TGF-β1 treatment impacts histologic staining of collagen, expression of key genes associated with fibroplasia, and size of the final contracted cell-ECM spheroids.

Final organization of deposited collagen. The most commonly used commercially-available method for quantification of deposited collagen is the Sircol™ insoluble collagen assay kit, which implements the dye Sirius Red F3B due to its high specificity for collagen. These kits, however, are optimized for use on fixed quantities of excised tissue. Due to the low assay volume and variability in assay final size (FIG. 7A), Sircol™ kits were not practical. Enzyme-linked immunosorbent assays (ELISAs) for soluble collagen fragments were also difficult to use due to the small amounts of material produced by the small number of cells and high background protein concentrations from the FBS-supplemented media.

Picrosirius red (PSR) utilizes the same anionic dye as Sircol™ assay kits to visualize collagen in paraffin embedded tissue sections. Under light microscopy, PSR stained collagen appears red and can be used for qualitative evaluation of collagen organization. A variety of quantitative approaches for morphometric assessment of collagen networks implement polarized light to visualize fiber alignment; however, signal strength and hue under linear polarized light are heavily dependent on sample orientation. Fluorescent imaging of PSR stained tissues with standard red filter sets yields a strong red fluorescence signal that is sensitive, collagen-specific, and is unaffected by sample orientation.

In order to evaluate deposited collagen, contracted cell-ECM spheroids were collected after 12 days of culture. Intermediate time points could not be sectioned due to adhesion of flat fibrin scaffolds to the microplate. Fluorescent micrographs demonstrate relatively homogenous collagen distribution for the interior of the contracted assay with higher deposition around the edge (FIG. 7A). Evaluation of the projected area demonstrated a TGF-β1 dose-dependent increase in final contracted spheroid size (P<0.05). Mean fluorescence intensity (MFI) was measured in order to evaluate relative differences in collagen organization between sections (FIG. 7B). While this measure cannot provide absolute quantification of collagen content, it indicated relative consistency in organization of collagen networks across different TGF-β1 conditions and the control.

Well-established mechanisms have linked TGF-β1 signaling to exaggerated extracellular deposition of type I collagen in fibrosis. Here, histologic evaluation indicates consistency in ECM deposition between conditions, suggesting that the conveniently visualized contracted spheroid size is correlated with the total amount of collagen accumulated during assay progression.

### Example 21: Alterations in mRNA Expression

In order to further evaluate the factors contributing to altered ECM remodeling with TGF-β1 stimulation, qPCR was used to determine mRNA expression for proteins involved in fibrinolysis and collagen deposition.

Quantification of mRNA for SERPENE1, which encodes for the protein plasminogen activator inhibitor type 1 (PAI-1), demonstrated significant time-dependent and dose-dependent increases in expression in response to TGF-β1 (FIG. 7C) similar to what has been reported in a prior fibrin fibroplasia assay. PAI-1 is the dominant inhibitor of fibrinolysis, and acts by binding to the active sites of urokinase-type and tissue-type plasminogen activators (uPA and tPA). These three regulators have been evaluated extensively in animal models of IPF to evaluate their potential involvement in fibrosis pathogenesis. TGF-β1 mediated increases in PAI-1 contribute to the anti-fibrinolytic environment during certain stages of wound healing and fibrosis. Additionally, gene polymorphisms of TGF-β1 and PAI-1 have been associated with susceptibility to IPF due in part to dysregulation of the fibrinolytic system.

Time course measurements also show significant increases in expression of the genes for tPA and uPA relative to the initial time point, but the effect of TGF-β1 stimulation is inverted between these two plasminogen activators. uPA demonstrated relative upregulation compared to the control time series, while tPA demonstrated a relative downregulation (FIGS. 7D and 7E). Other activators and inhibitors produced by cells can also impact conversion of plasminogen to plasmin. The phenotypic fibrin remodeling assay reflects the aggregate effects of these and other pathways. It is noted that while decreased fibrinolysis is one manifestation of increased PAI-1 levels, the mechanism by which it promotes fibroplasia may be through other pathways such as insulin-like growth factor binding protein 3 (IGFBP3).

In addition to its effects on the fibrinolytic system, TGF-β1 also has established roles in myofibroblast activation and collagen synthesis. Myofibroblasts are collagen-producing cells that express the contractile protein alpha smooth muscle actin (aSMA). Increases in myofibroblast activation and myofibroblast resistance to apoptosis have been identified as major contributors to IPF pathogenesis. Evaluation of ACTA2 mRNA demonstrated significant time-course increases in aSMA expression as well as increased expression for the highest concentration of TGF-β1 (FIG. 7F). These time-course changes may be due to a variety of factors including biomechanical feedback, cytokine secretion, or downstream signaling of the fibrinolytic system.

COL1A1 encodes the pro-alpha1(I) chain, which is a primary component of type I collagen. Quantification of mRNA for COL1A1 demonstrated dose-dependent increase in COL1A1 in response to TGF-β1 (FIG. 7G). Collagen expression in pulmonary fibrosis is heavily dependent on myofibroblast activation, but increased collagen expression in fibroblasts has also been linked to downstream effects of anti-fibrinolytic environments.

Expression of MKI67 mRNA was evaluated as a marker for proliferation. MKI67 expression was significantly upregulated with higher concentrations of TGF-β1, indicating increased cellular proliferation relative to the control condition (FIG. 7H). The initial decrease in MKI67 expression in all conditions indicates inhibition of proliferation by the fibrin scaffold, as compared to the cell suspension used for time point zero. Pulmonary fibroblasts have previously been shown to proliferate in response to TGF-β1. Additionally, the 0 ng/ml TGF-β1 conditions contracted within 24 hours, and this dense contracted matrix may have inhibited proliferation compared to TGF conditions which had not yet contracted.

TGF-β1 is a key regulator of ECM remodeling and dysregulation of TGF-β function is closely associated with fibrosis. The assay reveals multiple effects of TGF-β1 on fibroblasts including its ability to impact ECM remodeling through regulation of the fibrinolytic system and upregulated collagen synthesis.

### Example 22: Label-Free Quantification of Fibroplasia

In order to evaluate fibrosis in vitro, conventional approaches generally quantify specific contributors, such as activation of myofibroblasts or concentration of soluble collagen, using multi-step post-culture procedures. Here, the extent to which the unexpected, cell-driven ECM contraction and spheroid formation process could be used as a label-free approach was tested to assess fibroplasia.

An image processing pipeline was established in order to automate quantification and to allow consistent human bias-free analysis. Due to transitions in cell-ECM construct appearance over the course of the experiment, the built-in image segmentation software in the live-cell imager could not provide accurate segmentation. To overcome this image analysis challenge, Ilastic, a freely available image classification tool developed by the European Molecular Biology Laboratory was utilized.

Ilastik's pixel classification tool utilizes a random forest algorithm that can be interactively trained through iterations of user annotations on a small set of training images. To ensure consistent performance over the duration of the experiments, training images with a variety of features taken at different time points throughout the course of the assay were selected. When additional training annotations no longer improved background noise and edge feature fit, the trained pixel classification algorithm was saved for future use. Ilastik performs semantic segmentation, which returns probability maps that can be converted into masks by thresholding. In order to remove remaining background noise, opening and closing morphological filters were applied to the masks.

The projected area of the final contracted cell-ECM spheroid was the primary readout evaluated in the analysis, which demonstrated a significant dose-dependent increase with TGF-β1 stimulation (FIG. 8A). COL1A1 mRNA quantification and histologic analysis demonstrated increased collagen synthesis and deposition in response to TGF-β1. These data and observations demonstrate that greater spheroid size correlates with increased collagen deposition.

### Example 23: Evaluation of Serum and Cell Number Effects

The effects of serum concentration on matrix remodeling were evaluated by varying volumetric percentage of FBS in the cell culture media. FBS contains a complex mix of growth factors, hormones, cytokines, proteases, zymogens, co-factors, latent TGF-β1, and inhibitors that influence cellular activity. In the context of fibrin remodeling, an important component of FBS is plasminogen which can be activated by fibroblasts into plasmin for cell-mediated fibrinolysis. Assay media conditions ranging from serum-free to 8% FBS were evaluated. FBS-free conditions did not induce contraction within the duration of the experiments. In the absence of FBS, cell-ECM constructs also maintained their opaque appearance, indicating minimal fibrin degradation.

The projected-area of the final, contracted cell-ECM spheroids exhibited dose-dependent increases in response to FBS (FIG. 8B). These effects may be due, at least in part, to FBS components such as latent TGF-β1 and fibroblast growth factor (FGF). Increased fibroplasia in response to increasing concentrations of FBS is relevant to fibrotic disease. In vivo tissue availability of serum proteins depends largely on vascular permeability, and dysregulated endothelial permeability and vascular leak are associated with pulmonary fibrosis.

The initial fibroblast seeding density used in assays is also relevant to IPF. Fibroblasts from fibrotic lungs have particularly proliferative phenotypes, resulting in higher numbers of fibroblasts and myofibroblasts. Over a fibroblast seeding density range of between 1000 and 8000 cells/μl, a cell number-dependent increase was observed in the final projected cell-ECM spheroid area as expected (FIG. 8C).

### Example 24: Fibroblast Donor Variability

Prior studies have observed altered fibrogenic response in aged and diseased pulmonary fibroblasts compared to fibroblasts from younger and normal donors. PAI-1 production and TGF-β1 signaling have both been implicated in this pathogenic alteration in behavior. Primary human pulmonary fibroblasts from 2 normal donors and 2 IPF diseased donors were tested. For one of the normal donors, the PAI-1 production and TGF-β1 signaling were also compared at a higher passage (p11).

Sections of the final contracted cell-ECM spheroids showed consistency in organization of collagen (FIGS. 9A and 9B). The projected areas of the contracted cell-ECM spheroids in presence of FBS but without TGF-β1 was also relatively consistent across cells from different donors (FIG. 9I). The response to TGF-β1 addition, however, varied significantly (FIG. 9J). NHLFA showed significantly greater response to TGF-β1 compared to NHLFB. The high passage lineage of NHLFB showed an even smaller, statistically insignificant response. Despite both being considered normal, NHLFA was isolated from a 67-year-old male donor, while NHLFB came from a 79-year-old female smoker. Senescent phenotypes induced by age, smoking, and high-passage number may explain the varying responsiveness to TGF-β1 observed. Nintedanib treatment reduced the final projected area, although one of the two IPF fibroblasts did not reach statistical significance (p<0.3). In IPF donors, elevated in vivo exposure to TGF-β1 results in a heterogenous population of both fibroblasts and myofibroblasts. This heterogeneity may also contribute to variability in final contracted area.

### Example 25: Drug Response

Here, the response of lung fibroblasts from one normal and one IPF donor was tested using three different drugs that target different pathways (FIGS. 10A through 10D). Pirfenidone has been established to reduce fibroblast proliferation, α-SMA expression, and collagen synthesis. Nintedanib is a multiple tyrosine kinase inhibitor with effects on expression of ECM proteins and TGF-β1 induced signaling. Treatment with pirfenidone demonstrated a significant decrease in final area of TGF treated spheroids (P<0.01). Nintedanib conditions showed significant decreases in final area for all conditions (P<0.05). The ability of the contacting scar-in-a-drop assay described here to reveal anti-fibrotic effects of pirfenidone and nintedanib contrasts with the cell-mediated fibrinolysis assay described above that did not show significant effects of nintedanib and pirfenidone.

Nintedanib and pirfenidone are the two current FDA-approved therapeutics for IPF. In clinical use, however, these drugs do not halt or reverse fibrosis, and merely slow the progression of fibrotic scarring in the lungs. To address the need for alternative treatment strategies, several recent reviews have proposed components of the fibrinolytic system as potential targets for therapeutic intervention. Inhibition of PAI-1 is of particular interest, as its increased expression in IPF has been associated with worse clinical outcome. TM5275 is a small molecule inhibitor of PAI-1, which has been shown to minimize the extent of fibrotic remodeling in an animal model of pulmonary fibrosis and trigger apoptosis in TGF-β1 treated (but not untreated) fibroblasts and myofibroblasts. Consistent with these prior observations, TM5275 decreased the spheroid area for all the TGF-β1 treated conditions (P<0.05) but not in conditions that omitted TGF-β1. This difference in response may be related to PAI-1 upregulation with TGF-β1, where elevated levels could enable TM5275 to inhibit PAI-1 more effectively.

Fibrosis is the aggregate outcome of multiple dysregulated pathways. The ability of the contracting scar-in-a-drop assay to robustly detect effects of three different anti-fibrotic agents that work through disparate pathways, including the only 2 FDA-approved drugs, is encouraging for broader drug testing applications in the future.

Despite its importance in wound healing and fibrosis, fibrin gels have seen limited applications within fibroplasia assays. Instead, current prevailing assays focus on specific aspects of collagen production or cellular activation. FIG. 6D illustrates the steps of wound healing and shows where current phenotypic fibrosis assays stand.

Macromolecular crowding agent-based approaches, so-called scar-in-a-jar assays, have recently been the focus of several publications. The scar-in-a-jar assays are possible to perform in high throughput format but have difficulty detecting effects of some drugs when the readout is based on ECM production. The readouts are always based on methods that requires additional procedures such as staining or immunoassays. The ATPS bioprinting method uses macromolecules, particularly dextran, for ATPS-based fibrin printing; this may appear to mimic the scar-in-a-jar assay. The polymers in this assay, however, are quickly washed out after the 30 min cross-linking reaction, although the presence of some residual dextran is not completely ruled out. The assay is also different in allowing contraction of the cell-produced ECM.

Wound closure involves contraction at the macroscopic scale and fibrosis involves mechanical activation of cytokines and mechanotransduction making assays that provide readouts of mechanical function of cells important. The collagen contraction assay is the classic assay of this type and has been used extensively over the years. These assays, however, are also not sensitive to effects of anti-fibrotic drugs such as pirfenidone and the well-to-well variability can be quite large. Furthermore, collagen is known to inhibit fibroplasia by limiting collagen production by cells. This contracting scar-in-a-drop assay is beneficial in starting from a collagen-free gel to allow uninhibited cellular collagen deposition followed by contraction once fibrin has sufficiently degraded. This integrated approach enables this assay to replicate, in vitro, more of the biological wound healing process compared to prior models, showing the cumulative impact of multiple steps of an abnormal scarring process.

In summary, this paper reports a unique microscale fibrosis assay that induces fibroplasia in fibrin gels, uninhibited by presence of pre-existing collagen, that in later stages undergo a dramatic ECM contraction. The convenience of direct visual readouts of fibroplasia coupled with high sensitivity to multiple anti-fibrotic drugs makes this assay promising for drug testing applications. Given the variety of diseases that involve fibroplasia and mechano-transduction such as fibrotic diseases, cancer, and cardiovascular diseases; this phenotypic assay provides broad utility beyond IPF. This paper focuses on evaluating the projected area of cell-ECM spheroids after contraction. Given the central role of cell and tissue mechanics in fibrosis, there may also be opportunities to analyze contraction dynamics to gain additional information and readouts from this assay in the future.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A method for forming a microscale cell-laden matrix using an aqueous two-phase system (“ATPS”) comprising a mixture of a first material and a second material having a phase boundary between the first and second materials, the method comprising:
(a) mixing an enzyme with the first material;
(b) mixing a protein with the second material; and
(c) mixing a suspension comprising cells with one of the first material or second material;
wherein the enzyme, protein, and suspension comprising cells generate the cell-laden matrix;
wherein the first material is a first polymer selected from the group consisting of polyethylene glycol, polyvinyl pyrrolidone, polyvinyl alcohol, and ficoll, and the second material is a second polymer selected from the group consisting of dextran, polyvinyl pyrrolidone, polyvinyl alcohol, and ficoll; and
wherein the first material and the second material are different.

2. The method of claim 1, wherein at least one of the enzyme or the cells in the first material are configured to diffuse into the second material.

3. The method of claim 1, wherein the mixture comprises up to about 300 microliters (μL) of volume.

4. The method of claim 1, wherein the enzyme comprises a plasma enzyme from the group consisting of prothrombin, thrombin, amylase, pepsin, lipoprotein lipase, and pseudo-choline esterase.

5. The method of claim 1, wherein the protein comprises a plasma protein from the group consisting of fibrinogen, fibronectin, collagen, albumin, globulin, and plasminogen activator inhibitor type 1.

6. The method of claim 1, wherein the suspension comprising cells comprises fibroblasts, fibrocytes, osteoblasts, myofibroblasts, epithelial cells, endothelial cells, immune cells, mesenchymal cells, cancer cells, and stem cells.

7. The method of claim 1, further comprising mixing the mixture with a third material comprising one or more additives.

8. The method of claim 7, further comprising imaging the cell-laden matrix and the one or more additives.

9. The method of claim 7, wherein the one or more additives comprises transforming growth factor beta 1 (TGF-β1).

10. The method of claim 1, further comprising adding, to the cell-laden matrix, a digestive agent.

11. The method of claim 10, further comprising imaging the cell-laden matrix and the digestive agent.

12. The method of claim 1, further comprising detecting one or more remodeling events of the cell-laden matrix selected from the group consisting of matrix degradation, matrix growth, matrix proliferation, matrix cell invasion, matrix cell contraction, matrix cell type, and matrix cell density.

13. A cell-laden matrix assay system, comprising:
a solid support comprising at least one defined area; and
an aqueous two-phase system mixture for forming a cell-laden matrix, the mixture comprising:
a first material comprising an enzyme and one or more cells,
a second material comprising a protein, and
a phase boundary between the first and second materials;
wherein the first material is a first polymer selected from the group consisting of polyethylene glycol, polyvinyl pyrrolidone, polyvinyl alcohol, and ficoll, and the second material is a second polymer comprising dextran.

14. The system of claim 13, wherein the at least one defined area comprises up to about 300 microliters (μL) of volume.

15. The system of claim 13, wherein forming the cell-laden matrix comprises the enzyme, the protein, and at least one cell.

16. The system of claim 13, wherein said solid support is selected from the group consisting of a plate, a multiwell plate, a microfluidic device, and a slide.

17. The system of claim 13, further comprising a third material comprising one or more additives.

18. The system of claim 13, further comprising a digestive agent.

19. The system of claim 13, further comprising a detection system selected from the group consisting of label-free image processing, colorimetric, fluorescent, fluorescence polarization or lifetime readings, refractive index change, and electrochemical detection systems.

20. The system of claim 13, wherein:
the enzyme comprises a plasma enzyme from the group consisting of prothrombin, thrombin, amylase, pepsin, lipoprotein lipase, and pseudo-choline esterase;
the protein comprises a plasma protein from the group consisting of fibrinogen, albumin, globulin, and plasminogen activator inhibitor type 1; and
the one or more cells comprises a fibroblast, a fibrocyte, an osteoblast, a myofibroblast, an epithelial cell, a mesenchymal cell, a cancer cell, and a stem cell.
